SEQUENCE LISTING (7 sequences)

SEQ ID NO: 1 - protein, 7 aa, mammalian
RVYIHPF

SEQ ID NO: 2 - protein, 6 aa, mammalian
VYIHPF

SEQ ID NO: 3 - DNA, 21 bp, mammalian
ucgggcacagaucauuauutt

SEQ ID NO: 4 - DNA, 20 bp, mammalian
aacctgagtcaggacgtaat

SEQ ID NO: 5 - protein, 14 aa, mammalian
DRVYIHPFHLVHTG

SEQ ID NO: 6 - protein, 10 aa, mammalian
DRVYIHPFHL

SEQ ID NO: 7 - protein, 8 aa, mammalian
DRVYIHPF

(19) United States
(12) Patent Application Publication
(10) Pub. No.: US 2008/0026409 A1
(43) Pub. Date: Jan. 31, 2008

(54) SALT-SENSITIVE HYPERTENSION

(75) Inventors: Robert S. Danziger, Chicago, IL (US); Randal A. Skidgel, Winfield, IL (US)

Correspondence Address: AKERMAN SENTERFITT, P.O. BOX 3188, WEST PALM BEACH, FL, 33402-3188, US

(73) Assignee: THE BOARD OF TRUSTEES OF THE UNIVERSITY OF ILLINOIS, URBANA, IL (US)

(21) Appl. No.: 11/625,212

(22) Filed: Jan. 19, 2007

Related U.S. Application Data
(60) Provisional application No. 60/760,565, filed on Jan. 20, 2006.

(51) Int. Cl. G01N 33/53; C40B 30/06; C40B 40/10

(52) U.S. Cl. 435/7.8; 506/10; 506/18

(57) ABSTRACT

Identification of compositions of biomarkers which diagnose and distinguish between salt-sensitive hypertension, salt-resistant hypertension, salt-independent hypertension and/or hypertensive disorders comprise identifying varying levels of biomarkers in each patient as compared to normal patients. Methods for the identification of novel biomarkers and determination of minimal maximally effective doses of pharmaceutical agents used in treating patients are provided.

### CROSS-REFERENCE TO RELATED APPLICATION

The present application claims the priority of U.S. provisional patent application No. 60/760,565, entitled “URINE TEST FOR SALT-SENSITIVE HYPERTENSION,” filed Jan. 20, 2006, which is incorporated herein by reference in its entirety.

### STATEMENT AS TO FEDERALLY FUNDED RESEARCH

The present invention is funded in part by the National Institutes of Health grant No. 1R21DK065628-01. The U.S. government has certain rights in this invention.

### FIELD OF THE INVENTION

This invention relates to hypertension and disorders thereof.

### BACKGROUND

The epidemiologic, clinical, and experimental support for dietary sodium intake being one of the most important environmental determinants of human hypertension is overwhelming. First, the Intersalt Study, which recorded data from 52 sites around the world, showed that the risk of developing hypertension over 3 decades of adult life was linearly and very tightly related to 24-hour urine sodium excretion, the best measure of dietary sodium intake. Second, reduced dietary sodium intake and diuretics are among the most effective treatments for primary hypertension, and are currently recommended as first-line therapy in all the major practice guidelines around the world. However, both normotensive and hypertensive persons show tremendous inter-individual variability in their blood pressure responses to dietary sodium loading and sodium restriction. This inherent variability indicates strong genetic underpinnings. Third, the handful of rare Mendelian forms of human hypertension all involve excessive renal retention of salt and water, leading to severe salt-dependent hypertension. Fourth, numerous polygenetic rat models of salt dependent hypertension have been generated.

Affecting 50 million Americans and over 500 million people worldwide, hypertension is the most common treatable risk factor for death and disability from heart disease, stroke, and kidney failure. Fewer than one-fifth of the affected people have their pressures adequately controlled. The cause of most hypertension is unknown, thus, there is no cure. Dietary sodium intake is one of the most important environmental determinants of human hypertension. Novel insights into molecular mechanisms of salt-sensitivity and adaptation to salt may lead to new therapeutic targets and agents.

A general problem in the pharmacological therapies is determining the minimum effect dose of a medication. This is often difficult since measurements of biochemical efficacy are infrequently available. This is especially true with antihypertensive drugs in general, and specifically with renin and angiotensin converting enzyme (ACE) inhibitors.

A major problem in the management of hypertensive patients is the difficulty in identifying those that are salt-sensitive, i.e., those in whom hypertension is exacerbated by dietary salt and in whom salt-restriction is most beneficial and necessary. There is thus a need in the art for the diagnosis, risk analysis, determination of minimum effective doses of a drug and treatment of such individuals.

### SUMMARY

Identification of compositions of biomarkers which diagnose and distinguish between salt-sensitive hypertension, salt-resistant hypertension, salt-independent hypertension and/or hypertensive disorders comprise identifying varying levels of biomarkers in each patient as compared to normal patients. Methods for the identification of novel biomarkers and determination of minimal maximally effective doses of pharmaceutical agents used in treating patients are provided.

In a preferred embodiment, a method of detecting, diagnosing and differentiating between salt-sensitive hypertension, salt-resistant hypertension, salt-independent hypertension and/or hypertensive disorders comprises detecting at least one or more biomarkers in a subject sample, and; correlating the detection of one or more biomarkers with a salt-sensitive hypertension, salt-resistant hypertension, salt-independent hypertension and/or hypertensive disorders wherein the correlation takes into account the detection of one or more biomarker in each diagnosis, as compared to normal subjects wherein the one or more markers are selected from: APN, Ang II, Ang III, Ang IV, Ang 1-7, Anpep, aminopeptidase A (APA), AT4, Na/K ATPase, aldosterone, angiotensinogen, Mas and ACE2 and metabolites thereof; correlating the detection of one or more protein biomarkers with a diagnosis and differentiation between salt-sensitive hypertension, salt-resistant hypertension, salt-independent hypertension and/or hypertensive disorders, wherein the correlation takes into account the detection of one or more protein biomarkers in each diagnosis, as compared to normal subjects. Preferably, at least one of the biomarkers is detected, more preferably, a plurality of the biomarkers are detected. Any combination of biomarkers and metabolites thereof are used for detecting, diagnosing and differentiating between salt-sensitive hypertension, salt-resistant hypertension, salt-independent hypertension and/or hypertensive disorder.

In another preferred embodiment, the biomarkers further comprise metabolites of APN, Ang II, Ang III, Ang IV, Ang 1-7, Anpep, aminopeptidase A (APA), AT4, Na/K ATPase, angiotensinogen and ACE2.

In another embodiment, wherein biomarkers distinguishing salt-resistant hypertension from salt-independent and salt-sensitive hypertension comprise: APN, Ang II, Ang IV, Anpep, AT4 and Ang 1-7. For example, expression levels of biomarkers APN, Ang IV, Anpep, AT4 and Ang 1-7, Ang II, Ang III, and APA differ between biomarkers detected in salt-sensitive hypertensive patients. The detected biomarkers are compared between urine, plasma and tissue (e.g. renal cells) in patients from salt-sensitive, salt-resistant, salt-independent hypertension and normal individuals.

In another preferred embodiment, APN activity and Ang II metabolites are predictive of salt-sensitive hypertension.

In another preferred embodiment, a sample is selected from the group consisting of saliva, sputum, breath condensate, blood, blood plasma, serum, urine, tissue, cells, and liver. Preferably, the sample is urine and/or plasma.

In another preferred embodiment, biomarkers detected in a patient are elevated as compared to a normal healthy control.

In yet another embodiment, any combination of at least two biomarkers are used for detecting, diagnosing and differentiating between salt-sensitive hypertension, salt-resistant hypertension, salt-independent hypertension and/or hypertensive disorders.

In another embodiment, a plurality of the markers are used in combination for detecting, diagnosing and differentiating between salt-sensitive hypertension, salt-resistant hypertension, salt-independent hypertension and/or hypertensive disorders. One or more protein biomarkers are detected by comparing biomarker profiles from patients susceptible to, or suffering from salt-sensitive hypertension, salt-resistant hypertension, salt-independent hypertension and/or hypertensive disorders, with normal subjects.

In another preferred embodiment one or more protein biomarkers are detected using a fluorogenic assay immunoassay.

In another preferred embodiment, one or more protein biomarkers are detected using a biochip array.

In another preferred embodiment, a method of diagnosing salt-sensitive hypertension in a subject, comprising measuring at least one of: APN, Ang II, Ang III, Ang IV, Ang 1-7, Anpep, aminopeptidase A (APA), AT4, Na/K ATPase, aldosterone, angiotensinogen, Mas and ACE2, and/or metabolites thereof, in a biological sample obtained from said subject, wherein an elevated level of APN, Ang II Ang III, Ang IV, Ang 1-7, Anpep, aminopeptidase A (APA), AT4, Na/K ATPase, angiotensinogen, Mas and ACE2 and/or metabolites thereof, compared to normal subjects is diagnostic of salt-sensitive hypertension.

In another preferred embodiment, a method of monitoring effectiveness of treatment of salt-sensitive hypertension comprising measuring at least one of: APN, Ang II, Ang III, Ang IV, Ang 1-7, Anpep, aminopeptidase A (APA), AT4, Na/K ATPase, aldosterone, angiotensinogen, Mas and ACE2 and metabolites, thereof, in a biological sample obtained from said subject, wherein detected levels of APN, Ang III, Ang IV, Ang 1-7, Anpep, aminopeptidase A (APA), AT4, Na/K ATPase, aldosterone, angiotensinogen, Mas and ACE2 compared to normal subjects is indicative of the effectiveness of treatment of salt-sensitive hypertension.

In another embodiment, a kit for diagnosing and differentiating between salt-sensitive hypertension, salt-resistant hypertension, salt-independent hypertension and/or hypertensive disorders in a subject, the kit comprises (a) a substrate for holding a biological sample isolated from a human subject suspected of having hypertension, (b) a colorimetric agent that detects at least one or more biomarkers; (c) a panel of biomarkers; and, (d) printed instructions for reacting the agent with the biological sample or a portion of the biological sample to detect the presence or amount of at least one marker in the biological sample. Preferably, the panel of biomarkers comprise any one or more of: APN, Ang III, Ang IV, Ang 1-7, Anpep, aminopeptidase A (APA), AT4, Na/K ATPase, aldosterone, angiotensinogen, Mas and ACE2 and/or metabolites thereof.

In another embodiment, the kit further comprises antibodies specific for any one or more biomarkers: APN, Ang III, Ang IV, Ang 1-7, Anpep, aminopeptidase A (APA), AT4, Na/K ATPase, aldosterone, angiotensinogen, Mas and ACE2 and/or metabolites thereof.

In yet another embodiment, a method of identifying patients at risk of developing salt-sensitive hypertension comprises detecting at least one or more biomarkers in a subject sample, and; correlating the detection of one or more biomarkers with a diagnosis of salt-sensitive hypertension, wherein the correlation takes into account the detection of one or more biomarker in patients, as compared to normal subjects wherein the one or more markers are selected from: APN, Ang III, Ang IV, Ang 1-7, Anpep, aminopeptidase A (APA), AT4, Na/K ATPase, aldosterone, angiotensinogen, Mas and ACE2 and/or metabolites thereof; correlating the detection of one or more protein biomarkers with at risk patients for development of salt-sensitive hypertension, wherein the correlation takes into account the detection of one or more protein biomarkers in each at risk patient, as compared to normal subjects.

In another preferred embodiment, a panel of biomarkers comprises one or more biomarkers selected from: APN, Ang III, Ang IV, Ang 1-7, Anpep, aminopeptidase A (APA), AT4, Na/K ATPase, aldosterone, angiotensinogen, Mas and ACE2 and/or metabolites thereof.

In another preferred embodiment, a method of identifying therapeutically effective doses of a pharmaceutical agent for treating hypertension in a subject, comprising measuring at least one of: APN, Ang III, Ang IV, Ang 1-7, Anpep, aminopeptidase A (APA), AT4, Na/K ATPase, aldosterone, angiotensinogen, Mas and ACE2 and/or metabolites thereof in a biological sample obtained from said subject, wherein an elevated or decreased level of APN, Ang III, Ang IV, Ang 1-7, Anpep, aminopeptidase A (APA), AT4, Na/K ATPase, aldosterone, angiotensinogen, Mas and ACE2 and/or metabolites thereof, compared to normal subjects is predictive of a decrease in hypertension and identifies the therapeutic effective dose.

Other aspects are described infra.

### BRIEF DESCRIPTION OF DRAWINGS

The invention is pointed out with particularity in the appended claims. The above and further advantages of this invention may be better understood by referring to the following description taken in conjunction with the accompanying drawings, in which:

FIGS. 1A-1C are graphs showing renal APN transcript (FIG. 1A); protein (FIG. 1B), and activity (FIG. 1C) are greater in salt-resistant (Dahl SR/Jr) and unselected strains on 8% versus 0.3% NaCl but not in the salt-sensitive Dahl SS/Jr strain. Transcript by RT/PCR; protein by Western analysis; and activity by metabolism fluorogenic substrate Strains compared by ANOVA and change between strains by two-sided t-test. mean +/−SEM; n=number 4-22 rats in each group. * P<0.05. The data show that APN and Ang IV mediate adaptation to high NaCl is supported by the data showing that APN and AT4 receptors are distributed in PT and DT.

FIGS. 2A and 2B are scans of photographs showing the results from the immunohistochemical analysis of Dahl SR/Jr rat kidney for APN (FIG. 2A) and AT4 receptors (FIG. 2B) in PT (p) and DT (d). Brown is positive (standard immunoperoxidase-hematoxylin stain)-arrows. APN signal in general with apical>basolateral intensity. AT4 generally with greater abundance toward basolateral surface. glomeruli (g). Salt loading increases Ang IV and Ang III abundance in PT and DT in salt-adapting rats. Ang IV mediation of APN is supported by our data that Ang IV in PT and DT is greater in Dahl SR/Jr rats on 8% versus 0.3% NaCl.

FIG. 3A is a scan of a photograph showing results from an immunohistochemistry stain. Ang IV, and Ang III are distributed in proximal (PT) and distal (DT) nephrons. Ang IV expression is greater in PTs and DTs in Dahl SR/Jr rats on 8% versus 0.3% NaCl diet. Kidneys were removed from Dahl SR/Jr rats and immunhistochemistry performed using indirect immunoperoxidase with primary polyclonal Abs (positive staining-arrows). Ang IV is detected in proximal and distal nephrons with diffuse cytoplasmic staining signal for Ang III and IV were quantitated (QIHC) in PTs and DTs of rats on 8.0% and 0.3% NaCl diets for 10 days (graph, right panel). Ang IV abundance was the same in PTs and DTs and greater in rats on 8% versus 0.3% NaCl. Ang III is more abundant in DTs than PTs and greater in rats on 8% versus 0.3% NaCl (right panel). * P<0.05, 8% vs 0.3% NaCl. PTp (white bars); DT d (black bars); glomeruli g.

FIG. 4 shows siRNA to APN reduces both APN and Ang IV expression in LLC-PK cells, indicating that endogenous APN forms Ang IV. The top panel is a scan of photographs showing quantitative immunohistochemistry (QIH-C) for APN and Ang IV. Cells were grown in 4 well chamber plates. For siRNA, each well was incubated (37° C. for 5 hours) with 1 ml media containing 4 μl of Enhancer R, 4 μl of RNAi (2 μg), 8 μl of Transmessenger Reagent and 892 μl of optimem followed by 0.5 ml of optimem+20% FBS for 48 hours. The cells were fixed with 3.7% formaldehyde and stained using DakoCytomation staining kit. The amount of DAB staining was measured using Q-IHC. N=10 fields analyzed/transfection×2 transfections P<0.05 vs control. Upper panel: representative fields. Positive staining-arrows.

FIG. 5 is a graph showing APN and Ang IV reduce expression of basolateral Na/K ATPase in LLCPK1 cells and siRNA to AT4 receptor partially abolishes regulation Ang IV. Biotinylation and cell treatments were performed as described in the examples section which follows. siRNA to APN and siRNA to AT4 receptor increase while APN overexpression and Ang IV (10⁻⁹ M×20 hours) tend to reduce expression of basolateral Na/K ATPase. Representative autoradiograph of Na-K ATPase as detected by Western blot represented above bar-graph. Upper panel: representative autoradiograph of Na-K ATPase as detected by Western blot. Overexpression of APN( APN Over Exp) n=3 (except Ang IV, n=6) * P<0.05 compared to control paired t-test) Complementary measurements of Na/K ATPase expression by confocal microscopy-FIG. 5).

FIGS. 6A-6G are scans of photographs showing Na/K ATPase distribution in LLCPK1 cells by immuno fluorescence (complementary to FIG. 5): Confocal images from z-stacked permeabilized LLCPK1 cells probed for Na/K ATPase using monoclonal Ab against the alpha subunit of Na/K ATPase (1:100). FIG. 6A: Control. FIGS. 6B-G show cells with different treatments: FIG. 6B—Incubation with Ang IV (10 nM×12 hours), showing endocytosis and reduced membrane abundance of Na/K ATPase; FIG. 6C—APN overexpression showing endocytosis and reduced membrane abundance of Na/K ATPase; FIG. 6D—APN overexpression plus siRNA to AT4, showing that reducing AT4 partially blocks APN induced endocytosis of Na/K ATPase; Panel E—siRNA to AT4; Panel F—siRNA to APN; FIG. 6G—oubain (50 nM×12 hours) showing endocytosis (control). All images were generated with a Zeiss LSM 410 laser scanning confocal microscope at 100×. All settings were the same for all images obtained. Images are representative of experiments repeated twice. Arrows: endocytosis of Na/K ATPase.

FIG. 7 is a graph showing Na/K ATPase activity parallels changes in basolateral Na/K ATPase expression in LLCPK1 cells treated with siRNA to APN and AT4; Ang IV and oubain (control). n=1 (triplicate). siRNA to APN (siRNA-APN); siRNA to AT4 (siRNA-AT4); APN over-expression (APN o/e). Together, these experiments indicate that APN and Ang IV reduce Na/K ATPase expression through endocytosis and that suppressing the AT4 receptor attenuates the response, indicating that the APN signaling pathway is Ang IV dependent.

FIG. 8 is a graph showing Ang IV infusion reduces blood pressure (BP) in Dahl SS/Jr rats on 8% NaCl. Radiotelemetric blood pressure monitoring with 10 day Ang IV (7 pmol/minute) infusion (===). Systolic BP (peak) 157-188 mmHg. BP for each rat normalized to day 9, each BP averaged during 12 hour dark cycle (rats maintained 12 hours light/dark cycle). Day 0—surgical implantation of pressure transducer and infusion pump. n =4,- - - less than days 6-9 (initial infusion of Ang IV) and 16-24 (P<0.05).

FIG. 9 is a schematic representation to illustrate a possible mechanism for role of APN in adaptation to high salt.

FIG. 10 is a schematic illustration of RAS signaling Yellow: Traditional RAS Blue: Ang II metabolites; Green: Receptors for Ang II metabolites; Red: Relevant amino/carboxypeptidases; Aminopeptidase N (Anpep); Aminopeptidase -A (APA); Proly carboxypeptidase (PCP); prolyl endopeptidase (PEP); neutral endopeptidase (NEP); Angiotensin converting enzyme (ACE); Angiotensin (Ang).

FIG. 11 is a graph showing urinary aminopeptidase N activity measurements in salt-resistant (SR), salt-sensitive (SS), and spontaneously hypertensive (SHR) rats on 8% NaCl diets.

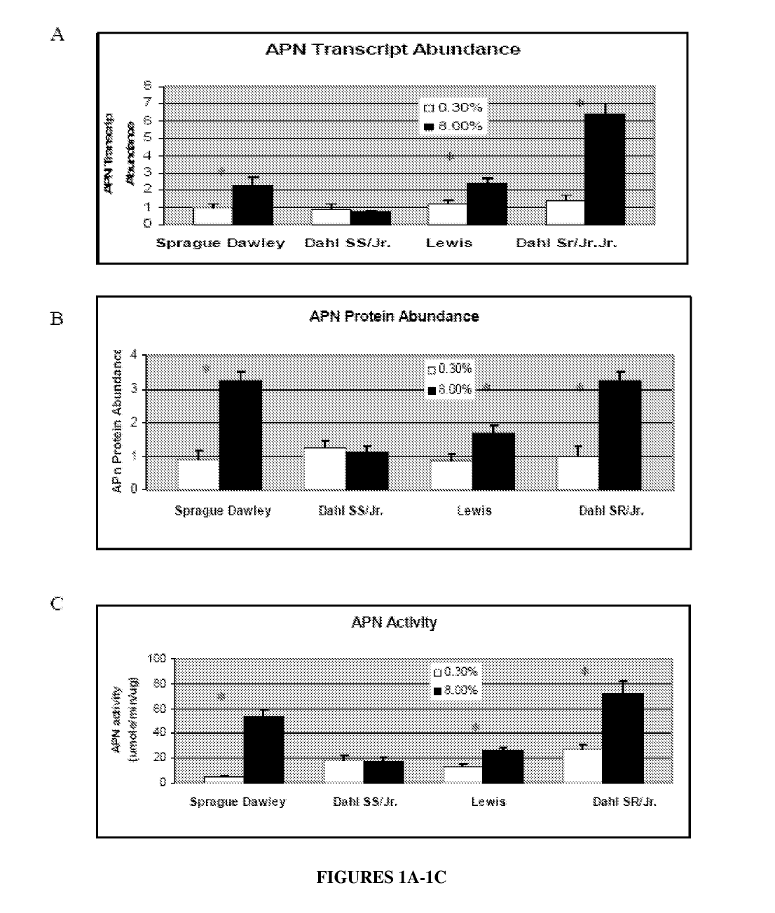

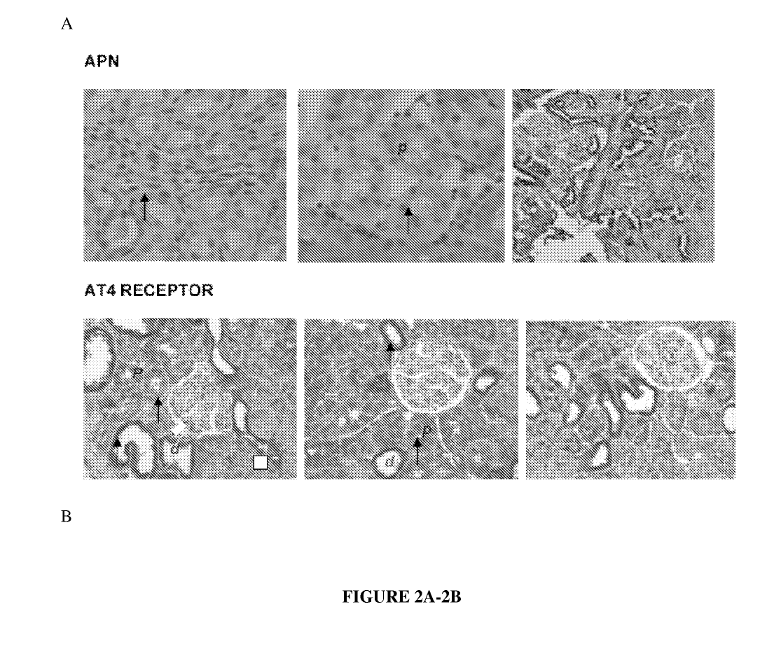

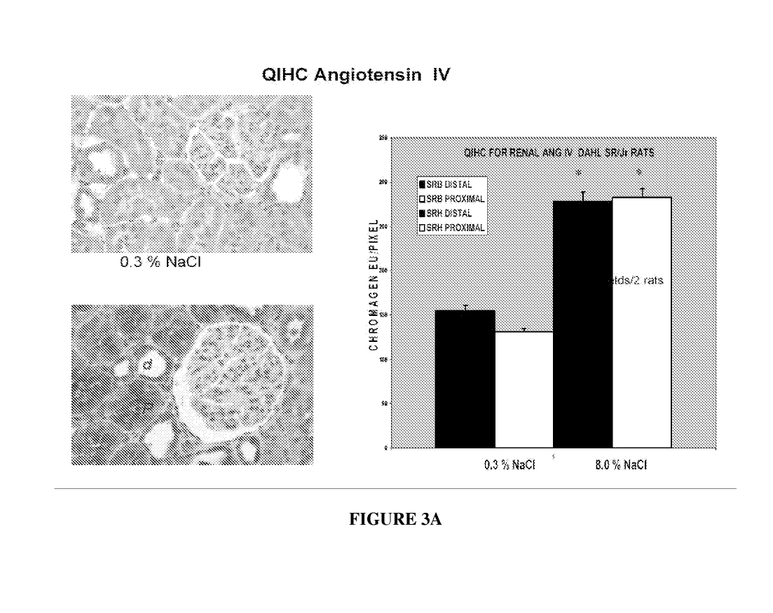

### DETAILED DESCRIPTION

A novel signaling pathway is identified. Compositions of biomarkers are identified which allow for the diagnosis and differentiation of different classes of hypertension. Methods relating to diagnosing and differentiating between salt-sensitive hypertension, salt-resistant hypertension, salt-independent hypertension and/or hypertensive disorders comprise identifying varying levels of biomarkers in each patient as compared to normal patients. Effective doses and types of pharmaceutical agents used in treating patients can be adapted based on the identity and levels of biomarkers present in the patient.

Mechanisms of adaptation to high NaCl: Although multiple signaling pathways, including nitric oxide, natriuretic peptides, endothelin, renal, endocrine, and vascular mechanisms, produce the salt-sensitive hypertensive phenotypes, an inability to increase Na excretion is central to each. Adaptation to high salt is mediated, at least in part, by increased abundance of the melanocortin receptor in the inner medullary collecting ducts; the transcriptional regulation of genes, including endothelial nitric oxide synthase (eNOS) and transforming growth factorβ1 (TGF β1; gamma-melanocyte-stimulating hormone gamma MSH) as well as changes in MAPK (particularly p38 MAPK and p42/44 MAPK). Other signaling pathways and proteins, e.g., Ang II, may play more subtle roles in regulation.

Determination of the role of renal tubule APN and Ang IV in adaptation to high salt; the establishment of renal tubule APN as a novel regulator of basolateral Na/K ATPase, and examination of APN signaling, focuses on Ang IV. This will define a novel and potentially important signaling pathway regulating Na uptake in adaptation to high salt.

Definitions

In accordance with the present invention and as used herein, the following terms are defined with the following meanings, unless explicitly stated otherwise.

As used herein, “a”, “an,” and “the” include plural references unless the context clearly dictates otherwise.

As used herein, the term “array” refers to an ordered spatial arrangement, particularly an arrangement of immobilized biomolecules.

As used herein, the term “addressable array” refers to an array wherein the individual elements have precisely defined x and y coordinates, so that a given element at a particular position in the array can be identified.

As used herein, the terms “probe” and “biomolecular probe” refer to a biomolecule used to detect a complementary biomolecule. Examples include antigens that detect antibodies, oligonucleotides that detect complimentary oligonucleotides, and ligands that detect receptors. Such probes are preferably immobilized on a microelectrode comprising a substrate.

As used herein, the terms “bioarray,” “biochip” and “biochip array” refer to an ordered spatial arrangement of immobilized biomolecules on a microelectrode arrayed on a solid supporting substrate. Preferred probe molecules include aptamers, nucleic acids, oligonucleotides, peptides, ligands, antibodies and antigens; peptides and proteins are the most preferred probe species. Biochips, as used in the art, encompass substrates containing arrays or microarrays, preferably ordered arrays and most preferably ordered, addressable arrays, of biological molecules that comprise one member of a biological binding pair. Typically, such arrays are oligonucleotide arrays comprising a nucleotide sequence that is complementary to at least one sequence that may be or is expected to be present in a biological sample. Alternatively, and preferably, proteins, peptides or other small molecules can be arrayed in such biochips for performing, inter alia, immunological analyses (wherein the arrayed molecules are antigens) or assaying biological receptors (wherein the arrayed molecules are ligands, agonists or antagonists of said receptors).

As used herein, the term “aptamer” or “selected nucleic acid binding species” shall include non-modified or chemically modified RNA or DNA. The method of selection may be by, but is not limited to, affinity chromatography and the method of amplification by reverse transcription (RT) or polymerase chain reaction (PCR).

As used herein, the term “fragment or segment”, as applied to a nucleic acid sequence, gene or polypeptide, will ordinarily be at least about 5 contiguous nucleic acid bases (for nucleic acid sequence or gene) or amino acids (for polypeptides), typically at least about 10 contiguous nucleic acid bases or amino acids, more typically at least about 20 contiguous nucleic acid bases or amino acids, usually at least about 30 contiguous nucleic acid bases or amino acids, preferably at least about 40 contiguous nucleic acid bases or amino acids, more preferably at least about 50 contiguous nucleic acid bases or amino acids, and even more preferably at least about 60 to 80 or more contiguous nucleic acid bases or amino acids in length. “Overlapping fragments” as used herein, refer to contiguous nucleic acid or peptide fragments which begin at the amino terminal end of a nucleic acid or protein and end at the carboxy terminal end of the nucleic acid or protein. Each nucleic acid or peptide fragment has at least about one contiguous nucleic acid or amino acid position in common with the next nucleic acid or peptide fragment, more preferably at least about three contiguous nucleic acid bases or amino acid positions in common, most preferably at least about ten contiguous nucleic acid bases amino acid positions in common.

“Biological samples” include solid and body fluid samples. The biological samples used in the present invention can include cells, protein or membrane extracts of cells, blood or biological fluids such as ascites fluid or brain fluid (e.g., cerebrospinal fluid). Examples of solid biological samples include, but are not limited to, samples taken from tissues of the central nervous system, bone, breast, kidney, cervix, endometrium, head/neck, gallbladder, parotid gland, prostate, pituitary gland, muscle, esophagus, stomach, small intestine, colon, liver, spleen, pancreas, thyroid, heart, lung, bladder, adipose, lymph node, uterus, ovary, adrenal gland, testes, tonsils and thymus. Examples of “body fluid samples” include, but are not limited to blood, serum, semen, prostate fluid, seminal fluid, urine, saliva, sputum, mucus, bone marrow, lymph, and tears.

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

“Marker” in the context of the present invention refers to a polypeptide (of a particular apparent molecular weight) which is differentially present in a sample taken from patients having a disease or disorder as compared to a comparable sample taken from control subjects (e.g., a person with a negative diagnosis, normal or healthy subject).

“Complementary” in the context of the present invention refers to detection of at least two biomarkers, which when detected together provides increased sensitivity and specificity as compared to detection of one biomarker alone.

The phrase “differentially present” refers to differences in the quantity and/or the frequency of a marker present in a sample taken from patients having for example, salt-sensitive hypertension as compared to a control subject. For example, a marker can be a polypeptide which is present at an elevated level or at a decreased level in samples of patients with hypertension (e.g. Ang II metabolites) compared to samples of control subjects. Alternatively, a marker can be a polypeptide which is detected at a higher frequency or at a lower frequency in samples of patients compared to samples of control subjects. A marker can be differentially present in terms of quantity, frequency or both.

A polypeptide is differentially present between the two samples if the amount of the polypeptide in one sample is statistically significantly different from the amount of the polypeptide in the other sample. For example, a polypeptide is differentially present between the two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polypeptide is differentially present between the two sets of samples if the frequency of detecting the polypeptide in samples of patients' suffering from disease or any disorder, is statistically significantly higher or lower than in the control samples. For example, a polypeptide is differentially present between the two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

“Diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

A “test amount” of a marker refers to an amount of a marker present in a sample being tested. A test amount can be either in absolute amount (e.g., μg/ml) or a relative amount (e.g., relative intensity of signals).

A “diagnostic amount” of a marker refers to an amount of a marker in a subject's sample that is consistent with a diagnosis of disease or any other disorder. A diagnostic amount can be either in absolute amount (e.g., μg/ml) or a relative amount (e.g., relative intensity of signals).

A “control amount” of a marker can be any amount or a range of amount which is to be compared against a test amount of a marker. For example, a control amount of a marker can be the amount of a marker in a person without disease or any other disorder. A control amount can be either in absolute amount (e.g., μg/ml) or a relative amount (e.g., relative intensity of signals).

“Probe” refers to a device that is removably insertable into a gas phase ion spectrometer and comprises a substrate having a surface for presenting a marker for detection. A probe can comprise a single substrate or a plurality of substrates.

“Substrate” or “probe substrate” refers to a solid phase onto which an adsorbent can be provided (e.g., by attachment, deposition, etc.).

“Adsorbent” refers to any material capable of adsorbing a marker. The term “adsorbent” is used herein to refer both to a single material (“monoplex adsorbent”) (e.g., a compound or functional group) to which the marker is exposed, and to a plurality of different materials (“multiplex adsorbent”) to which the marker is exposed. The adsorbent materials in a multiplex adsorbent are referred to as “adsorbent species.” For example, an addressable location on a probe substrate can comprise a multiplex adsorbent characterized by many different adsorbent species (e.g., anion exchange materials, metal chelators, or antibodies), having different binding characteristics. Substrate material itself can also contribute to adsorbing a marker and may be considered part of an “adsorbent.”

“Adsorption” or “retention” refers to the detectable binding between an absorbent and a marker either before or after washing with an eluant (selectivity threshold modifier) or a washing solution.

“Eluant” or “washing solution” refers to an agent that can be used to mediate adsorption of a marker to an adsorbent. Eluants and washing solutions are also referred to as “selectivity threshold modifiers.” Eluants and washing solutions can be used to wash and remove unbound materials from the probe substrate surface.

“Resolve,” “resolution,” or “resolution of marker” refers to the detection of at least one marker in a sample. Resolution includes the detection of a plurality of markers in a sample by separation and subsequent differential detection. Resolution does not require the complete separation of one or more markers from all other biomolecules in a mixture. Rather, any separation that allows the distinction between at least one marker and other biomolecules suffices.

“Gas phase ion spectrometer” refers to an apparatus that measures a parameter which can be translated into mass-to-charge ratios of ions formed when a sample is volatilized and ionized. Generally ions of interest bear a single charge, and mass-to-charge ratios are often simply referred to as mass. Gas phase ion spectrometers include, for example, mass spectrometers, ion mobility spectrometers, and total ion current measuring devices.

“Mass spectrometer” refers to a gas phase ion spectrometer that includes an inlet system, an ionization source, an ion optic assembly, a mass analyzer, and a detector.

“Laser desorption mass spectrometer” refers to a mass spectrometer which uses laser as means to desorb, volatilize, and ionize an analyte.

“Detect” refers to identifying the presence, absence or amount of the object to be detected.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

Biomarkers and Compositions thereof

Adaptation to high salt includes a coordinated reduction in Na uptake by PT and distal nephrons, including the cortical collecting duct (CCD). The basolateral Na/K ATPase is the primary mechanism for NaCl uptake in tubules, and a critical regulator of Na adaptation in nephrons. While Na/K ATPase plays the primary role in the proximal tubules, both epithelial Na channel (eNaC) activity and Na/K ATPase may play important roles in regulating sodium in the distal nephrons and CCD.

The levels of basolateral Na/K ATPase are tightly regulated. Downregulation of basolateral Na/K ATPase after high salt adaptation is linked directly to caveolae-mediated endocytosis. Endogenous cardiotonic substances, e.g., marinobufagenin, dopamine, insulin, and Ang II, play a mechanistic role in the regulation of basolateral Na/K ATPase expression, but probably do not completely account for endocytosis of basolateral Na/K ATPase in salt-adaptation. We have reported that renal APN abundance and activity are increased during adaptation to high salt (Farjah H. et al. Hypertension 43(2):282-285 (2004)), and have novel results that suggest that APN and Ang IV, which is formed from Ang III by APN, reduce Na/K ATPase expression (see, the examples which follow).

Kinase-mediated signaling regulates clathrin- and caveolae/raft-mediated endocytosis. The Ang IV-activated kinases, extracellular signal-regulated kinase p42/44 MAPK and p38 MAPK, mediate endocytosis through phosphorylation of both Ser-11 on the alpha subunit of Na/K ATPase and of the GDI:Rab5 complex. The role of these kinases in the endocytosis of Na/K ATPase is not known. The work described herein begin to address this gap in our knowledge.

The present discovery is based on surprising results showing that renal tubule APN and Ang IV increase during adaptation to high salt.

APN: APN (EC 3.4.11.2, ap-n), also known as microsomal aminopeptidase or CDl3, is a homodimeric, membrane-bound, zinc-dependent, particle-bound aminopeptidase that preferentially releases neutral amino acids from the N-terminal end of oligopeptides and has specificity similar to that of cytosolic leucine aminopeptidase. APN is a major constituent of the brush border membranes of kidney tubule cells and enterocytes. Among its reported substrates, which include kallidin, enkephalins, and somatostatin, is the heptapeptide Ang III, which is converted to Ang IV and, along with dipeptidylaminopeptidase, degrades Ang IV. We have reported that adaptation to a high salt diet in normotensive rat strains is associated with increased renal APN transcript/protein abundance and activity. The mechanistic importance in renal salt-adaptation is examined by renal APN protein expression in proximal and distal tubules (PT and DT, respectively), and examining regulation of basolateral Na/K ATPase by APN.

Angiotensin IV: Our results show that Ang IV levels in PT and DTs are greater in a salt-resistant of rat strain, i.e., Dahl SR/Jr, on an 8% NaCl diet than a 0.3% NaCl diet, consistent with our hypothesis that APN signaling occurs through Ang IV in adaptation to high salt. Ang IV and its receptor AT4, an insulin-regulated membrane aminopeptidase (IRAP) have primarily been studied in the context of neural function and inflammation. However, AT4 receptors exist in human PTs, DT/collecting duct, vascular smooth muscle, and endothelial cells. We have detected AT4 receptors throughout nephrons in the rat kidney. Ang IV and the AT4 receptor regulate several cellular functions, including stimulating endocytosis via Ras, p38 MAPK, and Erk-1/2, stimulating voltage-sensitive calcium and phosphorylating tyrosines in focal adhesion kinase (FAK) and paxillin.

Although Ang IV is a natriuretic peptide, its signaling pathway and role in salt-adaptation has not been established. A high-salt diet increases AT4 receptor expression in the PT of Wistar-Kyoto rats but not in spontaneously hypertensive rats (SHR). Ang IV infusion into the renal artery promotes Na excretion, increases cortical blood flow in the rat, and lowers blood pressure. In primary cultures of rat renal epithelial cells, Ang IV causes a dose-dependent decrease in transcellular Na transport. Our results show that Ang IV can reduce basolateral Na/K ATPase expression, the principal regulator of sodium uptake in the kidney.

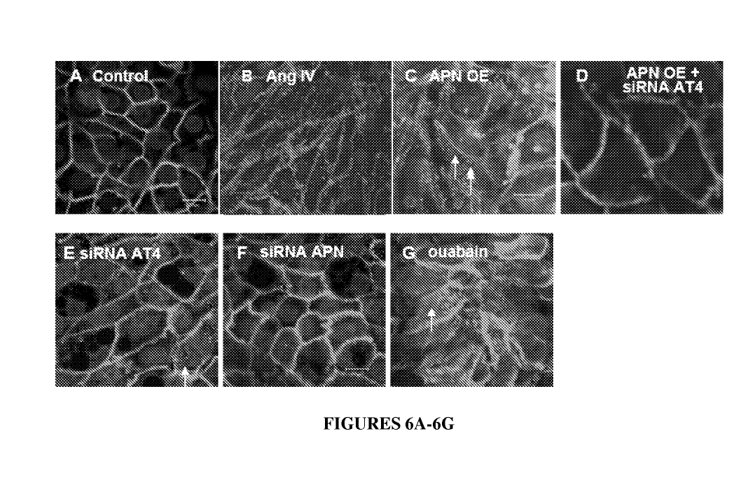

The results also show that Ang IV infusion lowers arterial blood pressure in Dahl SS/Jr rat on 8% NaCl (FIG. 6).

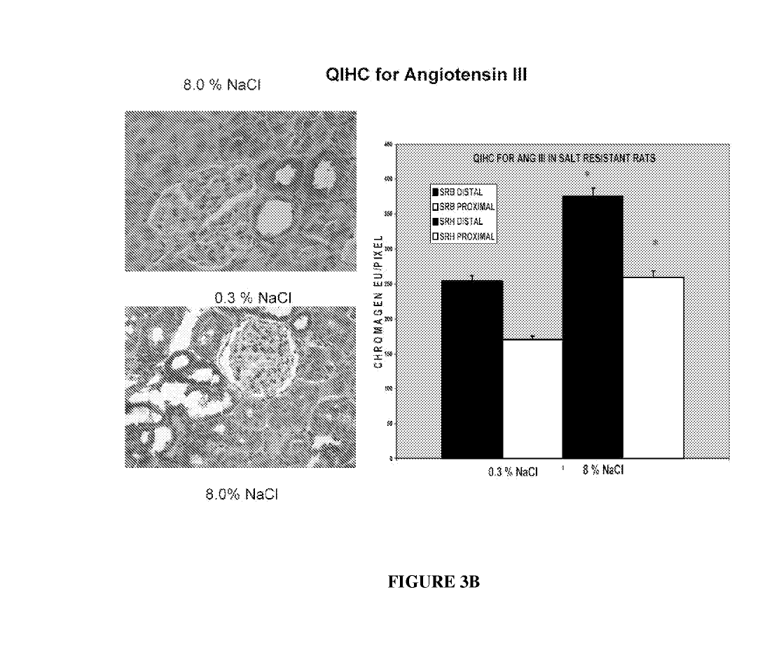

APN abundance regulates Ang IV: To initially address the hypothesis that the mechanism for APN-mediated regulation of Na/K ATPase is via Ang IV signaling, the effect of suppression of endogenous APN expression with siRNA on Ang IV abundance was measured in LLC-PK cells (FIG. 3A-3B). siRNA to APN decreased both APN and Ang IV expression, indicating the Ang IV level was regulated by APN. Therefore we find that the siRNA approach allows us to target APN specifically and demonstrate a significant effect on Ang IV expression.

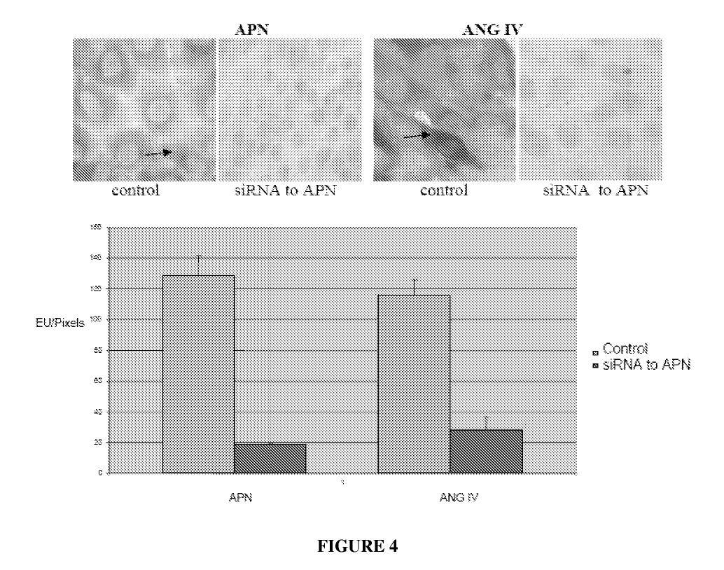

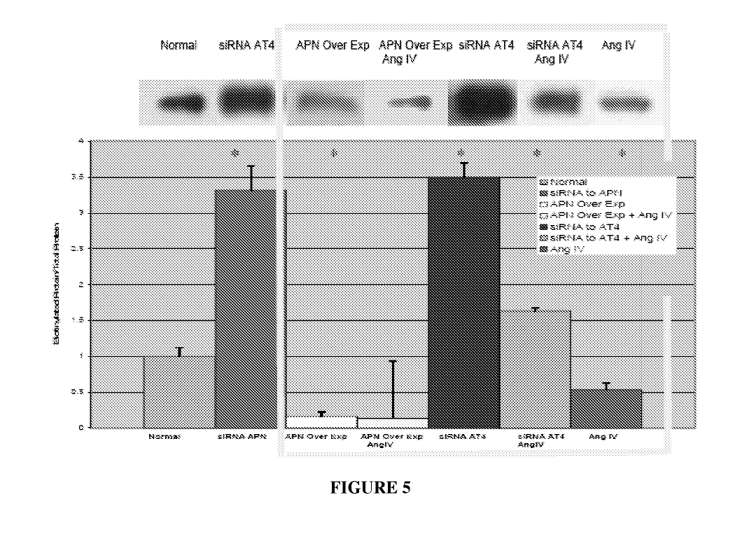

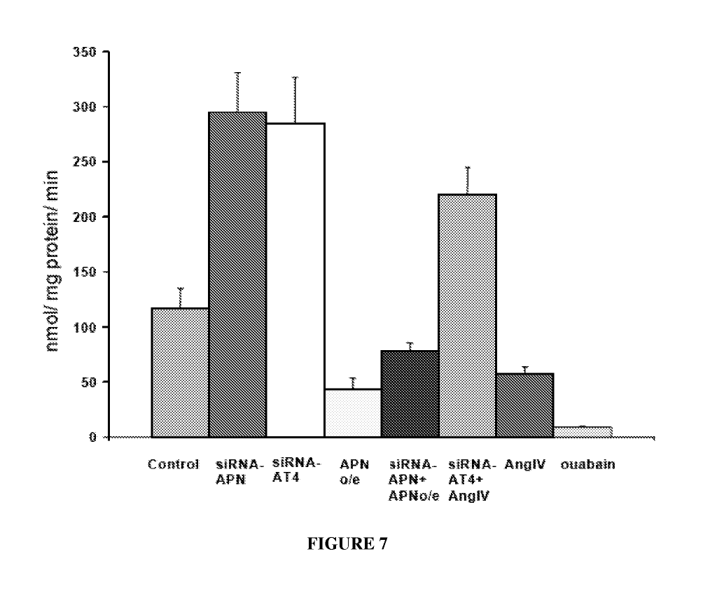

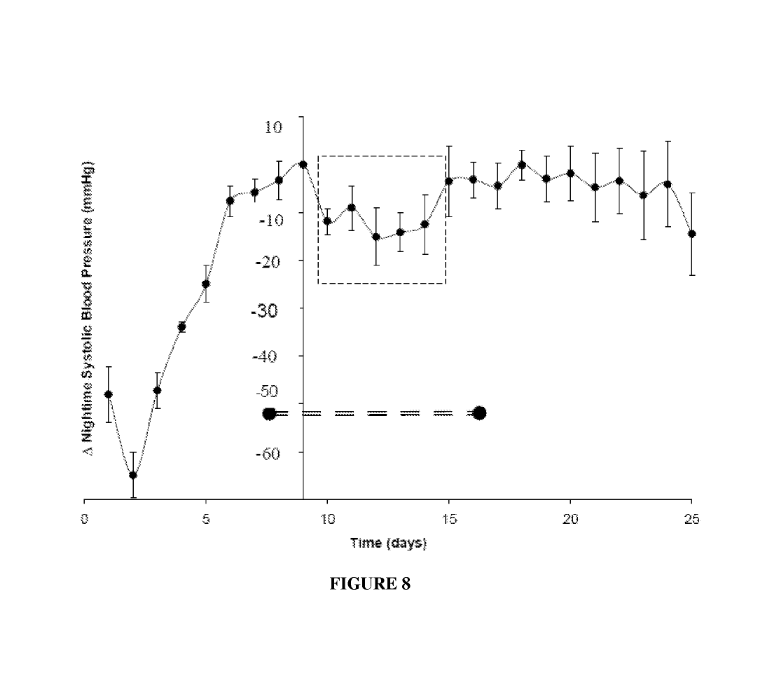

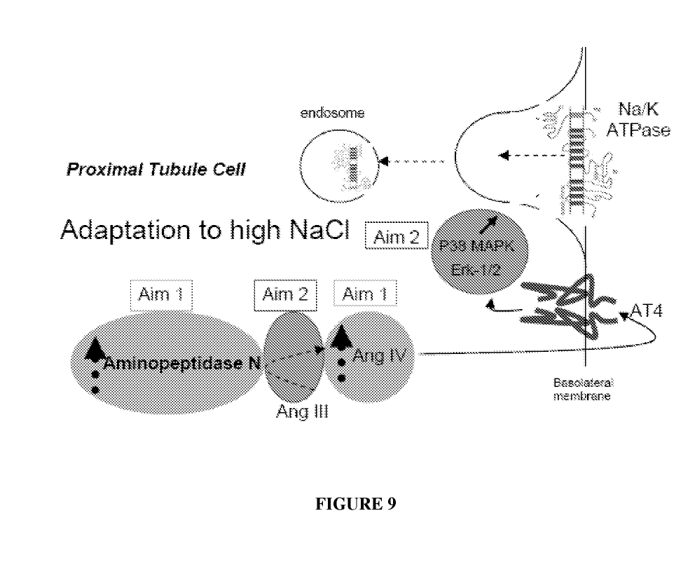

APN and Ang IV, via the AT4 receptor, reduce membrane expression (FIGS. 5, 6) and activity (FIG. 7) of basolateral Na/KATPase: To initially address the hypothesis that APN, Ang IV signaling, reduces basolateral Na/K ATPase abundance/activity, the effect of 1) over-expression of APN and 2) suppression of endogenous APN by siRNA on basolateral Na/K ATPase expression was measured in LLC-PK1 cell (FIG. 5). APN over-expression reduced, while siRNA to APN increased, basolateral Na/K ATPase expression, suggesting that Na/K ATPase expression is regulated by endogenous APN. Treatment of cells with Ang IV reduced Na/K ATPase expression both in the presence and absence of siRNA to APN. siRNA to AT4 receptor abrogated reduction in Na/K ATPase expression by Ang IV. However, confocal studies (FIG. 4; lower Panel 4D) suggest that siRNA to the AT4 receptor abrogrates endocytosis of Na/K ATPase by APN over-expression These findings indicat that Ang IV regulates Na/K ATPase expression and are consistent with APN-mediated regulation of Na/K ATPase expression via Ang IV signaling. Complementary confocal microscopy (FIG. 6) and activity measurements (FIG. 7).

In a preferred embodiment, compositions of biomarkers comprise angiotensin, angiotensin metabolites, aminopeptidases, carboxypeptidases, endopeptidases, variants, fragments, substrates and receptors thereof. Examples, include, but not limited to APN, Ang II, Ang III, Ang IV, Ang 1-7, Anpep, aminopeptidase A (APA), AT4, Na/K ATPase, aldosterone, Mas and ACE2.

In another preferred embodiment, the composition of biomarkers comprise any one or more metabolites of APN, Ang II, Ang III, Ang IV, Ang 1-7, Anpep, aminopeptidase A (APA), AT4, Na/K ATPase, aldosterone, Mas and ACE2.

In another preferred embodiment, urinary APN and Ang IV biomarkers distinguish between salt-sensitive and resistant forms of hypertension in patients.

In another preferred embodiment, an increase in expression of in renal tubule APN and Ang IV, DNA, RNA and expression products thereof in response to an intake of salt by the patient is indicative of resistance to salt-hypertension.

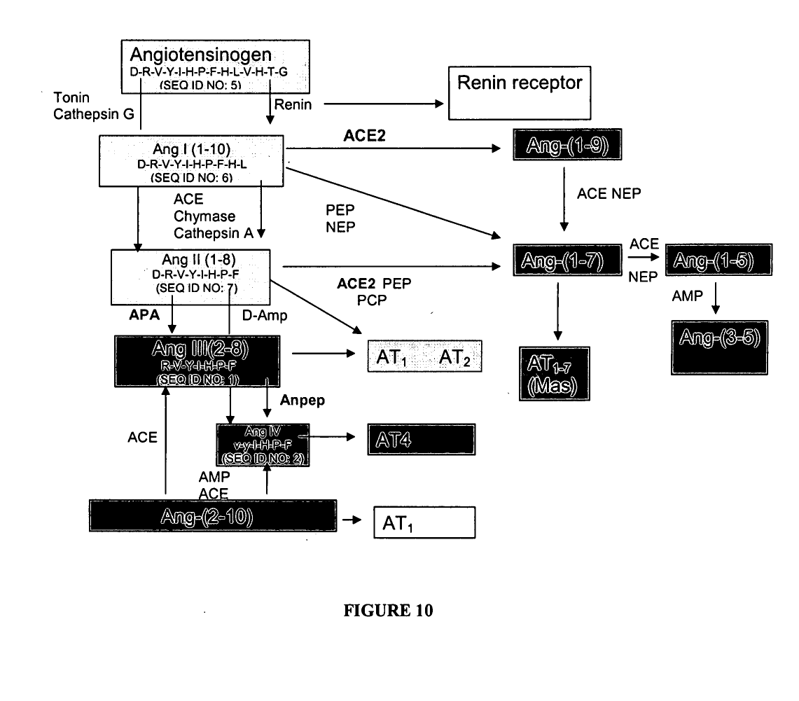

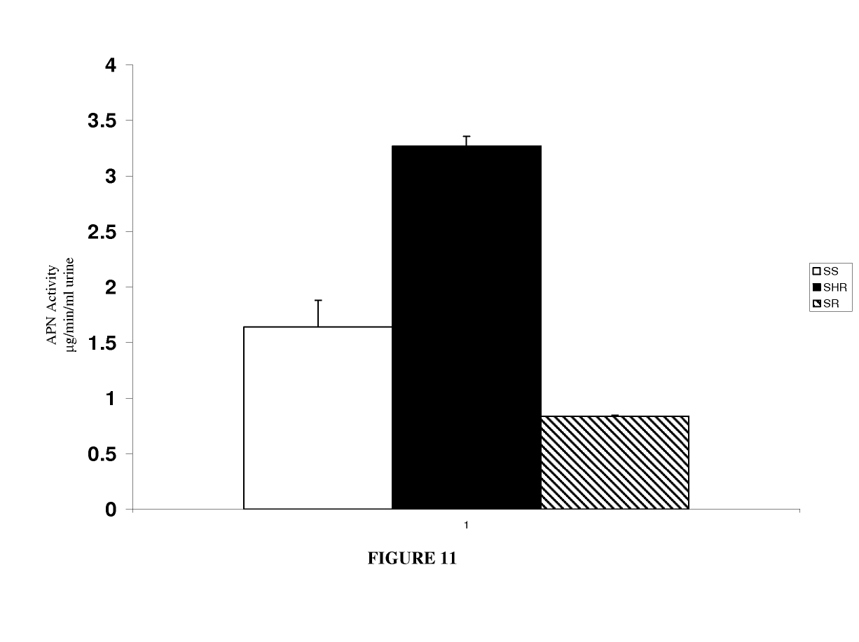

The renin-angiotensin-aldosterone system (RAS) plays a seminal role in the regulation of cardiovascular function in general and of blood pressure and renal salt-handling specifically. Angiotensin (Ang) II is generally considered the major physiological effector of RAS. However, Ang II metabolism and metabolites may form a previously unrecognized but critical component of RAS (see FIG. 10). However, a coordinated analysis of renal Ang II metabolism in hypertension and specifically in salt-adaptation in vivo is fundamental to understanding the role that angiotensin II metabolism and metabolites play in adaptation to high salt.

In a preferred embodiment, detection of one or more biomarkers is diagnostic for salt-sensitive hypertensive patients as compared to normal individuals.

In another preferred embodiment, detection of one or more biomarkers is a predictor of patients who are at risk for developing salt-sensitive hypertension.

In another preferred embodiment, detection of one or more biomarkers or grouping of biomarkers distinguish and identify patients who are salt-sensitive hypertensive versus salt-resistant hypertensive and/or salt-independent hypertensive. For example, detection of angiotensin, angiotensin metabolites, aminopeptidases, carboxypeptidases, endopeptidases, variants, fragments, substrates and receptors thereof.

In another preferred embodiment, detection of biomarkers that are differentially present in an individual are diagnostic of hypertensive disorders. Examples of biomarkers include, but not limited to: angiotensin, angiotensin metabolites, aminopeptidases, carboxypeptidases, endopeptidases, metabolites, variants, fragments, substrates and receptors thereof.

In another preferred embodiment, the invention provides for the quantitative detection of biomarkers diagnostic of salt-sensitive, salt-resistance and/or salt-independent hypertension and disorders thereof. Depending on the type and severity of disease biomarkers are differentially present and the ratios of these biomarkers are indicative of salt-sensitive hypertensive versus salt-resistant hypertensive and/or salt-independent hypertensive. For example, the ratios of APN, Ang III, Ang IV, Ang 1-7, Anpep, aminopeptidase A (APA), AT4, Na/K ATPase, aldosterone, Mas and ACE2, as compared to healthy individuals.

In another preferred embodiment, detection of certain biomarkers are diagnostic of the specific type of hypertension. For example, salt-sensitive hypertensive versus salt-resistant hypertensive versus salt-independent hypertensive.

In another preferred embodiment, salt-sensitive hypertensive versus salt-resistant hypertensive and/or salt-independent hypertensive and/or hypertensive disorders in a subject is analyzed by (a) providing a biological sample isolated from a subject suspected of having hypertension; (b) detecting in the sample the presence or amount of at least one marker selected from one or more biomarker proteins; and (c) correlating the presence or amount of the marker with the presence or type of hypertension in the subject. Preferably, a sample from a hypertensive individual, such as urine, serum, plasma, kidney cells, and the like, in in vitro culture or in situ in an animal subjects express higher levels and ratios of specific biomarkers as compared to non-hypertensive individuals. In one aspect of the invention, the samples comprise cells, for example, a biopsy of kidney tissue. In addition, biomarkers are detected in the circulating blood and other biofluids (e.g. urine, sweat, s saliva, etc.). Thus, other suitable biological samples include, but not limited to such cells or fluid secreted from these cells. Obtaining biological fluids such as blood, plasma, serum, saliva and urine, from a subject is typically much less invasive and traumatizing than obtaining a solid tissue biopsy sample. Thus, samples, which are biological fluids, are preferred for use in the invention.

A biological sample can be obtained from a subject by conventional techniques. Blood can be obtained by venipuncture, while plasma and serum can be obtained by fractionating whole blood according to known methods. Surgical techniques for obtaining solid tissue samples are well known in the art.

Any animal that expresses the hypertensive biomarker proteins, such as for example, APN, Ang III, Ang IV, Ang 1-7, Anpep, aminopeptidase A (APA), AT4, Na/K ATPase, aldosterone, Mas and ACE2, can be used as a subject from which a biological sample is obtained. Preferably, the subject is a mammal, such as for example, a human, dog, cat, horse, cow, pig, sheep, goat, primate, rat, or mouse. More preferably, the subject is a human. Particularly preferred are subjects suspected of having or at risk for developing salt-sensitive hypertension and related hypertensive disorders.

The biomarkers of the invention can be detected in a sample by any means. Methods for detecting the biomarkers are described in the materials and methods and Examples which follow. For example, fluorogenic assays, immunoassays, which include, but are not limited to: competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, fluorescent immunoassays and the like. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding an antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen (i.e. diabetic biomarker), coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

Enzymatic activity assays or fluorogenic assays based on fluorescence are comprised of fluorogenic peptide substrates designed such that the fluorescence changes when the substrate is metabolized to its product. One skilled in the art would be knowledgeable as to the design and specific parameters to optimize these types of assays. For further discussion regarding the use of fluorogenic assays see, e.g., Chahroudi A, Silvestri G, Feinberg MB. Methods. 2003 Oct;31(2):120-6. and Rust L et al. Methods Enzymol. 1994;235:554-62, incorporated herein by reference.

Identification of New Markers

In a preferred embodiment, a biological sample is obtained from a patient suffering from or susceptible to hypertension. Biological samples comprising biomarkers from other patients and control subjects (i.e. normal healthy individuals of similar age, sex, physical condition) are used as comparisons. Biological samples are extracted as discussed above. Preferably, the sample is prepared prior to detection of biomarkers. Typically, preparation involves fractionation of the sample and collection of fractions determined to contain the biomarkers. Methods of pre-fractionation include, for example, size exclusion chromatography, ion exchange chromatography, heparin chromatography, affinity chromatography, sequential extraction, gel electrophoresis and liquid chromatography. The analytes also may be modified prior to detection. These methods are useful to simplify the sample for further analysis. For example, it can be useful to remove high abundance proteins, such as albumin, from blood before analysis.

In one embodiment, a sample can be pre-fractionated according to size of proteins in a sample using size exclusion chromatography. For a biological sample wherein the amount of sample available is small, preferably a size selection spin column is used. In general, the first fraction that is eluted from the column (“fraction 1”) has the highest percentage of high molecular weight proteins; fraction 2 has a lower percentage of high molecular weight proteins; fraction 3 has even a lower percentage of high molecular weight proteins; fraction 4 has the lowest amount of large proteins; and so on. Each fraction can then be analyzed by immunoassays, gas phase ion spectrometry, and the like, for the detection of markers.

In another embodiment, a sample can be pre-fractionated by anion exchange chromatography. Anion exchange chromatography allows pre-fractionation of the proteins in a sample roughly according to their charge characteristics. For example, a Q anion-exchange resin can be used (e.g., Q HyperD F, Biosepra), and a sample can be sequentially eluted with eluants having different pH's. Anion exchange chromatography allows separation of biomarkers in a sample that are more negatively charged from other types of biomarkers. Proteins that are eluted with an eluant having a high pH is likely to be weakly negatively charged, and a fraction that is eluted with an eluant having a low pH is likely to be strongly negatively charged. Thus, in addition to reducing complexity of a sample, anion exchange chromatography separates proteins according to their binding characteristics.

In yet another embodiment, a sample can be pre-fractionated by heparin chromatography. Heparin chromatography allows pre-fractionation of the markers in a sample also on the basis of affinity interaction with heparin and charge characteristics. Heparin, a sulfated mucopolysaccharide, will bind markers with positively charged moieties and a sample can be sequentially eluted with eluants having different pH's or salt concentrations. Markers eluted with an eluant having a low pH are more likely to be weakly positively charged. Markers eluted with an eluant having a high pH are more likely to be strongly positively charged. Thus, heparin chromatography also reduces the complexity of a sample and separates markers according to their binding characteristics.

In yet another embodiment, a sample can be pre-fractionated by isolating proteins that have a specific characteristic, e.g. are glycosylated. For example, a blood, or serum sample can be fractionated by passing the sample over a lectin chromatography column (which has a high affinity for sugars). Glycosylated proteins will bind to the lectin column and non-glycosylated proteins will pass through the flow through. Glycosylated proteins are then eluted from the lectin column with an eluant containing a sugar, e.g., N-acetyl-glucosamine and are available for further analysis.

Thus there are many ways to reduce the complexity of a sample based on the binding properties of the proteins in the sample, or the characteristics of the proteins in the sample.

In yet another embodiment, a sample can be fractionated using a sequential extraction protocol. In sequential extraction, a sample is exposed to a series of adsorbents to extract different types of biomarkers from a sample. For example, a sample is applied to a first adsorbent to extract certain proteins, and an eluant containing non-adsorbent proteins (i.e., proteins that did not bind to the first adsorbent) is collected. Then, the fraction is exposed to a second adsorbent. This further extracts various proteins from the fraction. This second fraction is then exposed to a third adsorbent, and so on.

Any suitable materials and methods can be used to perform sequential extraction of a sample. For example, a series of spin columns comprising different adsorbents can be used. In another example, a multi-well comprising different adsorbents at its bottom can be used. In another example, sequential extraction can be performed on a probe adapted for use in a gas phase ion spectrometer, wherein the probe surface comprises adsorbents for binding biomarkers. In this embodiment, the sample is applied to a first adsorbent on the probe, which is subsequently washed with an eluant. Markers that do not bind to the first adsorbent are removed with an eluant. The markers that are in the fraction can be applied to a second adsorbent on the probe, and so forth. The advantage of performing sequential extraction on a gas phase ion spectrometer probe is that markers that bind to various adsorbents at every stage of the sequential extraction protocol can be analyzed directly using a gas phase ion spectrometer.

In yet another embodiment, biomarkers in a sample can be separated by high-resolution electrophoresis, e.g., one or two-dimensional gel electrophoresis. A fraction containing a marker can be isolated and further analyzed by gas phase ion spectrometry. Preferably, two-dimensional gel electrophoresis is used to generate two-dimensional array of spots of biomarkers, including one or more markers. See, e.g., Jungblut and Thiede, Mass Spectr. Rev. 16:145-162 (1997).

The two-dimensional gel electrophoresis can be performed using methods known in the art. See, e.g., Deutscher ed., Methods In Enzymology vol. 182. Typically, biomarkers in a sample are separated by, e.g., isoelectric focusing, during which biomarkers in a sample are separated in a pH gradient until they reach a spot where their net charge is zero (i.e., isoelectric point). This first separation step results in one-dimensional array of biomarkers. The biomarkers in one dimensional array is further separated using a technique generally distinct from that used in the first separation step. For example, in the second dimension, biomarkers separated by isoelectric focusing are further separated using a polyacrylamide gel, such as polyacrylamide gel electrophoresis in the presence of sodium dodecyl sulfate (SDS-PAGE). SDS-PAGE gel allows further separation based on molecular mass of biomarkers. Typically, two-dimensional gel electrophoresis can separate chemically different biomarkers in the molecular mass range from 1000-200,000 Da within complex mixtures.

Biomarkers in the two-dimensional array can be detected using any suitable methods known in the art. For example, biomarkers in a gel can be labeled or stained (e.g., Coomassie Blue or silver staining). If gel electrophoresis generates spots that correspond to the molecular weight of one or more markers of the invention, the spot can be further analyzed by densitometric analysis or gas phase ion spectrometry. For example, spots can be excised from the gel and analyzed by gas phase ion spectrometry. Alternatively, the gel containing biomarkers can be transferred to an inert membrane by applying an electric field. Then a spot on the membrane that approximately corresponds to the molecular weight of a marker can be analyzed by gas phase ion spectrometry. In gas phase ion spectrometry, the spots can be analyzed using any suitable techniques, such as MALDI or SELDI.

Prior to gas phase ion spectrometry analysis, it may be desirable to cleave biomarkers in the spot into smaller fragments using cleaving reagents, such as proteases (e.g., trypsin). The digestion of biomarkers into small fragments provides a mass fingerprint of the biomarkers in the spot, which can be used to determine the identity of markers if desired.

In yet another embodiment, high performance liquid chromatography (HPLC) can be used to separate a mixture of biomarkers in a sample based on their different physical properties, such as polarity, charge and size. HPLC instruments typically consist of a reservoir of mobile phase, a pump, an injector, a separation column, and a detector. Biomarkers in a sample are separated by injecting an aliquot of the sample onto the column. Different biomarkers in the mixture pass through the column at different rates due to differences in their partitioning behavior between the mobile liquid phase and the stationary phase. A fraction that corresponds to the molecular weight and/or physical properties of one or more markers can be collected. The fraction can then be analyzed by gas phase ion spectrometry to detect markers.

Optionally, a marker can be modified before analysis to improve its resolution or to determine its identity. For example, the markers may be subject to proteolytic digestion before analysis. Any protease can be used. Proteases, such as trypsin, that are likely to cleave the markers into a discrete number of fragments are particularly useful. The fragments that result from digestion function as a fingerprint for the markers, thereby enabling their detection indirectly. This is particularly useful where there are markers with similar molecular masses that might be confused for the marker in question. Also, proteolytic fragmentation is useful for high molecular weight markers because smaller markers are more easily resolved by mass spectrometry. In another example, biomarkers can be modified to improve detection resolution. For instance, neuraminidase can be used to remove terminal sialic acid residues from glycoproteins to improve binding to an anionic adsorbent and to improve detection resolution. In another example, the markers can be modified by the attachment of a tag of particular molecular weight that specifically bind to molecular markers, further distinguishing them. Optionally, after detecting such modified markers, the identity of the markers can be further determined by matching the physical and chemical characteristics of the modified markers in a protein database (e.g., SwissProt).

After preparation, biomarkers in a sample are typically captured on a substrate for detection. Traditional substrates include antibody-coated 96-well plates or nitrocellulose membranes that are subsequently probed for the presence of proteins. Preferably, the biomarkers are identified using immunoassays as described above. However, preferred methods also include the use of biochips, chromatography and the like. Preferably the biochips are protein biochips for capture and detection of proteins. Many protein biochips are described in the art. These include, for example, protein biochips produced by Packard BioScience Company (Meriden Conn.), Zyomyx (Hayward, Calif.) and Phylos (Lexington, Mass.). In general, protein biochips comprise a substrate having a surface. A capture reagent or adsorbent is attached to the surface of the substrate. Frequently, the surface comprises a plurality of addressable locations, each of which location has the capture reagent bound there. The capture reagent can be a biological molecule, such as a polypeptide or a nucleic acid, which captures other biomarkers in a specific manner. Alternatively, the capture reagent can be a chromatographic material, such as an anion exchange material or a hydrophilic material. Examples of such protein biochips are described in the for example in U.S. Pat. No. 6,225,047 (Hutchens and Yip, “Use of retentate chromatography to generate difference maps,” May 1, 2001).

In general, a sample containing the biomarkers is placed on the active surface of a biochip for a sufficient time to allow binding. Then, unbound molecules are washed from the surface using a suitable eluant. In general, the more stringent the eluant, the more tightly the proteins must be bound to be retained after the wash. The retained protein biomarkers now can be detected by appropriate means.

Analytes captured on the surface of a protein biochip can be detected by any method known in the art. This includes, for example, mass spectrometry, fluorescence, surface plasmon resonance, ellipsometry and atomic force microscopy. Mass spectrometry, and particularly SELDI mass spectrometry, is a particularly useful method for detection of the biomarkers of this invention.

A laser desorption time-of-flight mass spectrometer can be used in embodiments of the invention. In laser desorption mass spectrometry, a substrate or a probe comprising markers is introduced into an inlet system. The markers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

Matrix-assisted laser desorption/ionization mass spectrometry, or MALDI-MS, is a method of mass spectrometry that involves the use of an energy absorbing molecule, frequently called a matrix, for desorbing proteins intact from a probe surface. MALDI is described, for example, in U.S. Pat. No. 5,118,937 (Hillenkamp et al.) and U.S. Pat. No. 5,045,694 (Beavis and Chait). In MALDI-MS the sample is typically mixed with a matrix material and placed on the surface of an inert probe. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in this art. The matrix dries, forming crystals that encapsulate the analyte molecules. Then the analyte molecules are detected by laser desorption/ionization mass spectrometry. MALDI-MS is useful for detecting the biomarkers of this invention if the complexity of a sample has been substantially reduced using the preparation methods described above.

Surface-enhanced laser desorption/ionization mass spectrometry, or SELDI-MS represents an improvement over MALDI for the fractionation and detection of biomolecules, such as proteins, in complex mixtures. SELDI is a method of mass spectrometry in which biomolecules, such as proteins, are captured on the surface of a protein biochip using capture reagents that are bound there. Typically, non-bound molecules are washed from the probe surface before interrogation. SELDI is described, for example, in: U.S. Pat. No. 5,719,060 (“Method and Apparatus for Desorption and Ionization of Analytes,” Hutchens and Yip, Feb. 17, 1998,) U.S. Pat. No. 6,225,047 (“Use of Retentate Chromatography to Generate Difference Maps,” Hutchens and Yip, May 1, 2001) and Weinberger et al., “Time-of-flight mass spectrometry,” in Encyclopedia of Analytical Chemistry, R. A. Meyers, ed., pp 11915-11918 John Wiley & Sons Chichesher, 2000.

Markers on the substrate surface can be desorbed and ionized using gas phase ion spectrometry. Any suitable gas phase ion spectrometers can be used as long as it allows markers on the substrate to be resolved. Preferably, gas phase ion spectrometers allow quantitation of markers.

In one embodiment, a gas phase ion spectrometer is a mass spectrometer. In a typical mass spectrometer, a substrate or a probe comprising markers on its surface is introduced into an inlet system of the mass spectrometer. The markers are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of markers or other substances will typically involve detection of signal intensity. This, in turn, can reflect the quantity and character of markers bound to the substrate. Any of the components of a mass spectrometer (e.g., a desorption source, a mass analyzer, a detector, etc.) can be combined with other suitable components described herein or others known in the art in embodiments of the invention.

In another embodiment, an immunoassay can be used to detect and analyze markers in a sample. This method comprises: (a) providing an antibody that specifically binds to a marker; (b) contacting a sample with the antibody; and (c) detecting the presence of a complex of the antibody bound to the marker in the sample.

To prepare an antibody that specifically binds to a marker, purified markers or their nucleic acid sequences can be used. Nucleic acid and amino acid sequences for markers can be obtained by further characterization of these markers. For example, each marker can be peptide mapped with a number of enzymes (e.g., trypsin, V8 protease, etc.). The molecular weights of digestion fragments from each marker can be used to search the databases, such as SwissProt database, for sequences that will match the molecular weights of digestion fragments generated by various enzymes. Using this method, the nucleic acid and amino acid sequences of other markers can be identified if these markers are known proteins in the databases.

Alternatively, the proteins can be sequenced using protein ladder sequencing. Protein ladders can be generated by, for example, fragmenting the molecules and subjecting fragments to enzymatic digestion or other methods that sequentially remove a single amino acid from the end of the fragment. Methods of preparing protein ladders are described, for example, in International Publication WO 93/24834 (Chait et al.) and U.S. Pat. No. 5,792,664 (Chait et al.). The ladder is then analyzed by mass spectrometry. The difference in the masses of the ladder fragments identify the amino acid removed from the end of the molecule.

If the markers are not known proteins in the databases, nucleic acid and amino acid sequences can be determined with knowledge of even a portion of the amino acid sequence of the marker. For example, degenerate probes can be made based on the N-terminal amino acid sequence of the marker. These probes can then be used to screen a genomic or cDNA library created from a sample from which a marker was initially detected. The positive clones can be identified, amplified, and their recombinant DNA sequences can be subcloned using techniques which are well known. See, e.g., Current Protocols for Molecular Biology (Ausubel et al., Green Publishing Assoc. and Wiley-Interscience 1989) and Molecular Cloning: A Laboratory Manual, 3rd Ed. (Sambrook et al., Cold Spring Harbor Laboratory, NY 2001).

Using the purified markers or their nucleic acid sequences, antibodies that specifically bind to a marker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). Such techniques include, but are not limited to, antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

After the antibody is provided, a marker can be detected and/or quantified using any of suitable immunological binding assays known in the art (see, e.g., U.S. Pat. Nos. 4,366,241; 4,376,110; 4,517,288; and 4,837,168). Useful assays include, for example, an enzyme immune assay (EIA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asai, ed. 1993); Basic and Clinical Immunology (Stites & Terr, eds., 7th ed. 1991); and Harlow & Lane, supra.

Generally, a sample obtained from a subject can be contacted with the antibody that specifically binds the marker. Optionally, the antibody can be fixed to a solid support to facilitate washing and subsequent isolation of the complex, prior to contacting the antibody with a sample. Examples of solid supports include glass or plastic in the form of, e.g., a microtiter plate, a stick, a bead, or a microbead. Antibodies can also be attached to a probe substrate or ProteinChip® array described above. The sample is preferably a biological fluid sample taken from a subject. Examples of biological fluid samples include cerebrospinal fluid, blood, serum, plasma, urine, tears, saliva etc. In a preferred embodiment, the biological fluid comprises serum. The sample can be diluted with a suitable eluant before contacting the sample to the antibody.

After incubating the sample with antibodies, the mixture is washed and the antibody-marker complex formed can be detected. This can be accomplished by incubating the washed mixture with a detection reagent. This detection reagent may be, e.g., a second antibody which is labeled with a detectable label. Exemplary detectable labels include magnetic beads (e.g., DYNABEADS™), fluorescent dyes, radiolabels, enzymes (e.g., horse radish peroxide, alkaline phosphatase and others commonly used in an ELISA), and colorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker is incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays are carried out at ambient temperature, although they can be conducted over a range of temperatures, such as 10° C. to 40° C.

Immunoassays can be used to determine presence or absence of a marker in a sample as well as the quantity of a marker in a sample. First, a test amount of a marker in a sample can be detected using the immunoassay methods described above. If a marker is present in the sample, it will form an antibody-marker complex with an antibody that specifically binds the marker under suitable incubation conditions described above. The amount of an antibody-marker complex can be determined by comparing to a standard. A standard can be, e.g., a known compound or another protein known to be present in a sample. As noted above, the test amount of marker need not be measured in absolute units, as long as the unit of measurement can be compared to a control.

The methods for detecting these markers in a sample have many applications. For example, one or more markers (e.g. APN, Ang III, Ang IV, Ang 1-7, Anpep, aminopeptidase A (APA), AT4, Na/K ATPase, aldosterone, Mas and ACE2) can be measured to aid in the diagnosis of salt-sensitive hypertensive versus salt-resistant hypertensive versus salt-independent hypertensive and/or hypertensive disorders. In another example, the methods for detection of the markers can be used to monitor responses in a subject to treatment and effective doses of a drug used to treat such patients. In another example, the methods for detecting markers can be used to assay for and to identify compounds that modulate expression of these markers in vivo or in vitro.

Data generated by desorption and detection of markers can be analyzed using any suitable means. In one embodiment, data is analyzed with the use of a programmable digital computer. The computer program generally contains a readable medium that stores codes. Certain code can be devoted to memory that includes the location of each feature on a probe, the identity of the adsorbent at that feature and the elution conditions used to wash the adsorbent. The computer also contains code that receives as input, data on the strength of the signal at various molecular masses received from a particular addressable location on the probe. This data can indicate the number of markers detected, including the strength of the signal generated by each marker.

Data analysis can include the steps of determining signal strength (e.g., height of peaks) of a marker detected and removing “outliers” (data deviating from a predetermined statistical distribution). The observed peaks can be normalized, a process whereby the height of each peak relative to some reference is calculated. For example, a reference can be background noise generated by instrument and chemicals (e.g., energy absorbing molecule) which is set as zero in the scale. Then the signal strength detected for each marker or other biomolecules can be displayed in the form of relative intensities in the scale desired (e.g., 100). Alternatively, a standard (e.g., a serum protein) may be admitted with the sample so that a peak from the standard can be used as a reference to calculate relative intensities of the signals observed for each marker or other markers detected.

The computer can transform the resulting data into various formats for displaying. In one format, referred to as “spectrum view or retentate map,” a standard spectral view can be displayed, wherein the view depicts the quantity of marker reaching the detector at each particular molecular weight. In another format, referred to as “peak map,” only the peak height and mass information are retained from the spectrum view, yielding a cleaner image and enabling markers with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique markers and markers which are up- or down-regulated between samples. Marker profiles (spectra) from any two samples may be compared visually. In yet another format, Spotfire Scatter Plot can be used, wherein markers that are detected are plotted as a dot in a plot, wherein one axis of the plot represents the apparent molecular mass of the markers detected and another axis represents the signal intensity of markers detected. For each sample, markers that are detected and the amount of markers present in the sample can be saved in a computer readable medium. This data can then be compared to a control (e.g., a profile or quantity of markers detected in control, e.g., normal, healthy subjects in whom diabetes injury is undetectable).

Diagnosis and Differentiation between Salt-Sensitive, Salt-Resistant, and Salt-Independent Hypertension

In another aspect, the invention provides methods for diagnosing a salt-sensitive hypertensive versus salt-resistant hypertensive versus salt-independent hypertensive and/or hypertensive disorders using one or more markers. For example, proteins identified from patients comprising angiotensin, angiotensin metabolites, aminopeptidases, carboxypeptidases, endopeptidases, variants, fragments, substrates and receptors thereof. These markers can be used singularly or in combination with other markers in any set. The markers are differentially present in samples of a human patient, for example a salt-sensitive hypertension patient, and a normal subject in whom hypertension is undetectable. For example, some of the markers are expressed at an elevated level and/or are present at a higher frequency in human patients with salt-sensitive hypertensive versus salt-resistant hypertensive versus salt-independent hypertensive and/or hypertensive disorders than in normal subjects. Therefore, detection of one or more of these markers in a person would provide useful information regarding the probability that the person may have salt-sensitive hypertension versus salt-resistant hypertension versus salt-independent hypertension and/or hypertensive disorders. Examples of hypertensive biomarkers include, but not limited to angiotensin, angiotensin metabolites, aminopeptidases, carboxypeptidases, endopeptidases, variants, fragments, substrates and receptors or any combination thereof. Specific examples, include: APN, Ang III, Ang IV, Ang 1-7, Anpep, aminopeptidase A (APA), AT4, Na/K ATPase, aldosterone, Mas and ACE2 or any combination thereof.

Accordingly, embodiments of the invention include methods for aiding in a salt-sensitive hypertensive versus salt-resistant hypertensive versus salt-independent hypertensive and/or hypertensive disorder diagnosis, using one or more markers, wherein the method comprises: (a) detecting at least one marker in a sample, wherein the marker is APN, Ang III, Ang IV, Ang 1-7, Anpep, aminopeptidase A (APA), AT4, Na/K ATPase, aldosterone, Mas and ACE2, fragments, derivatives or variants thereof; and (b) correlating the detection of the marker or markers with a probable diagnosis of salt-sensitive hypertensive versus salt-resistant hypertensive versus salt-independent hypertensive and/or hypertensive disorders. The correlation may take into account the amount of the marker or markers in the sample compared to a control amount of the marker or markers (up or down regulation of the marker or markers) (e.g., in normal subjects in whom diabetes is undetectable). The correlation may take into account the presence or absence of the markers in a test sample and the frequency of detection of the same markers in a control. The correlation may take into account both of such factors to facilitate determination of whether a subject has salt-sensitive hypertensive versus salt-resistant hypertensive versus salt-independent hypertensive and/or hypertensive disorders and the degree of severity of the disease, or not.

Any suitable samples can be obtained from a subject to detect markers. Preferably, a sample is a urine sample from the subject. Preferably, a sample is tested for the presence of a plurality of markers. Detecting the presence of a plurality of markers, rather than a single marker alone, would provide more information for the diagnostician. Specifically, the detection of a plurality of markers in a sample would increase the percentage of true positive and true negative diagnoses and would decrease the percentage of false positive or false negative diagnoses.

The detection of the marker or markers is then correlated with a probable diagnosis of salt-sensitive hypertensive versus salt-resistant hypertensive versus salt-independent hypertensive and/or hypertensive disorders. In some embodiments, the detection of the mere presence or absence of a marker, without quantifying the amount of marker, is useful and can be correlated with a probable diagnosis of salt-sensitive hypertensive versus salt-resistant hypertensive versus salt-independent hypertensive and/or hypertensive disorders.

In other embodiments, the detection of markers can involve quantifying the markers to correlate the detection of markers with a probable diagnosis of salt-sensitive hypertensive versus salt-resistant hypertensive versus salt-independent hypertensive and/or hypertensive disorders, degree of severity of and the like. Thus, if the amount of the markers detected in a subject being tested is higher compared to a control amount, then the subject being tested has a higher probability of having salt-sensitive hypertensive versus salt-resistant hypertensive versus salt-independent hypertensive and/or hypertensive disorders.

Similarly, in another embodiment, the detection of markers can further involve quantifying the markers to correlate the detection of markers with a probable diagnosis of, degree of severity of salt-sensitive hypertensive versus salt-resistant hypertensive versus salt-independent hypertensive and/or hypertensive disorders and the like, wherein the markers are present in lower quantities in urine, blood serum etc, samples from patients than in urine samples of normal subjects. Thus, if the amount of the markers detected in a subject being tested is lower compared to a control amount, then the subject being tested has a higher probability of having salt-sensitive hypertensive versus salt-resistant hypertensive versus salt-independent hypertensive and/or hypertensive disorders.

When the markers are quantified, it can be compared to a control. A control can be, e.g., the average or median amount of marker present in comparable samples of normal subjects in whom salt-sensitive hypertensive versus salt-resistant hypertensive versus salt-independent hypertensive and/or hypertensive disorders, is undetectable. The control amount is measured under the same or substantially similar experimental conditions as in measuring the test amount. For example, if a test sample is obtained from a subject's urine sample and a marker is detected using a particular probe, then a control amount of the marker is preferably determined from a urine sample of a patient using the same probe. It is preferred that the control amount of marker is determined based upon a significant number of samples from normal subjects who do not have salt-sensitive hypertensive versus salt-resistant hypertensive versus salt-independent hypertensive and/or hypertensive disorders so that it reflects variations of the marker amounts in that population. By the same logic, markers found in salt-sensitive hypertensive versus salt-resistant hypertensive versus salt-independent hypertensive patients will produce differing biomarker profiles between these groups of patients.

Data generated by mass spectrometry can then be analyzed by a computer software. The software can comprise code that converts signal from the mass spectrometer into computer readable form. The software also can include code that applies an algorithm to the analysis of the signal to determine whether the signal represents a “peak” in the signal corresponding to a marker of this invention, or other useful markers. The software also can include code that executes an algorithm that compares signal from a test sample to a typical signal characteristic of “normal” and human salt-sensitive hypertensive versus salt-resistant hypertensive versus salt-independent hypertensive and/or hypertensive disorders and determines the closeness of fit between the two signals. The software also can include code indicating which the test sample is closest to, thereby providing a probable diagnosis.

Effective Dosing

A general problem in the pharmacological therapies is determining the minimum effect dose of a medication. This is often difficult since measurements of biochemical efficacy are infrequently available. This is especially true with antihypertensive drugs in general, and specifically with renin and angiotensin converting enzyme (ACE) inhibitors.

In a preferred embodiment, methods of identifying a minimum effective dose of a therapeutic agent in a patient suffering from hypertension comprises identifying biomarkers which are modulated in response to a dose of the agent. For example, in a salt-sensitive hypertensive patient, the dose of a therapeutic agent is varied until the expression of biomarkers are comparable to a normal individual. Thus, the doses can be individually tailored to the patient.

In another preferred embodiment, the minimal maximally effect dose of a renin and angiotensin converting enzyme (ACE) inhibitor is determined by measuring urinary and/or plasma biomarkers, e.g., but not limited to, angiotensinogen, aldosterone, Ang II, Ang III, Ang IV, Ang 1-7. When these biomarkers are of normal values or did not change with higher doses of the inhibitor, this indicates maximally effective pharmacologic dose.

Diagnostics, Candidate Therapeutic Compounds and Compositions

In another preferred embodiment, a method of identifying candidate therapeutic compounds comprises culturing cells expressing at least one marker selected from: APN, Ang III, Ang IV, Ang 1-7, Anpep, aminopeptidase A (APA), AT4, Na/K ATPase, aldosterone, Mas and ACE2 or any combination thereof, and identify those candidate compounds which bind to at least one of these markers.

In another preferred embodiment, candidate therapeutic compounds are identified based on the regulation of NA/KATPase expression/activity, and AT4 receptor expression/activity. Examples of measuring the expression/activity of these molecules is detailed in the examples section which follows.

A number of suitable assay methods to detect binding of test compounds to APN, Ang III, Ang IV, Ang 1-7, Anpep, angiotensinogen, aminopeptidase A (APA), AT4, Na/K ATPase, aldosterone, Mas and ACE2 or any combination thereof are known in the art, and include, but are not limited to, surface plasmon resonance (SPR)/Biacore™, fluorogenic binding assays, fluid phase binding assays, affinity chromatography, size exclusion or gel filtration, ELISA, immunoprecipitation, competitive binding assays, gel shift assays, and mass spectrometry based methods, inter alia.

In some embodiments, methods described herein include a first screen for compounds that bind to APN, Ang III, Ang IV, Ang 1-7, Anpep, aminopeptidase A (APA), AT4, Na/K ATPase, aldosterone, Mas and ACE2 or any combination thereof. Compounds that are identified as binding to APN, Ang III, Ang IV, Ang 1-7, Anpep, aminopeptidase A (APA), AT4, Na/K ATPase, aldosterone, Mas and ACE2 or any combination thereof can then be used in a second screen to identify those compounds that inhibit a function of APN, Ang III, Ang IV, Ang 1-7, Anpep, aminopeptidase A (APA), AT4, Na/K ATPase, aldosterone, Mas and ACE2 or any combination thereof. Alternatively, the first screen can be omitted and the compounds can simply be screened for their ability to inhibit a function of APN, Ang II, Ang III, Ang IV, Ang 1-7, Anpep, aminopeptidase A (APA), AT4, Na/K ATPase, aldosterone, Mas and ACE2 or any combination thereof.

Once a compound that inhibits an action of APN, Ang II, Ang III, Ang IV, Ang 1-7, Anpep, aminopeptidase A (APA), AT4, Na/K ATPase, aldosterone, Mas and ACE2 is identified, the compound can be considered a candidate compound that inhibits hypertension The ability of such compounds to treat hypertension can be evaluated in a population of viable cells or in an animal, e.g., an animal model. (See, for example, the methods which follow)

The methods include administering the compound to a model of the condition, e.g., contacting a cell (in vitro) model with the compound, or administering the compound to an animal model of the condition,

Test Compounds: The test compounds utilized in the assays and methods described herein can be, inter alia, nucleic acids, small molecules, organic or inorganic compounds, antibodies or antigen-binding fragments thereof, polynucleotides, peptides, or polypeptides. For example, APN, Ang III, Ang IV, Ang 1-7, Anpep, aminopeptidase A (APA), AT4, Na/K ATPase, aldosterone, Mas and ACE2 or any combination thereof, polypeptide variants including truncation mutants, deletion mutants, and point mutants; nucleic acids including sense, antisense, aptamers, and small inhibitory RNAs (siRNAs) including short hairpin RNAs (shRNAs) and ribozymes) can be used as test compounds in the methods described herein. Alternatively, compounds or compositions that mimic the binding portions of APN, Ang III, Ang IV, Ang 1-7, Anpep, aminopeptidase A (APA), AT4, Na/K ATPase, aldosterone, Mas and ACE2 or any combination thereof can be used. Candidate therapeutic agents, once screened in a clinical setting, are therapeutic agents, and both types of agents can be optionally optimized (e.g., by derivatization), and formulated with pharmaceutically acceptable excipients or carriers to form pharmaceutical compositions.

Small Molecules: Small molecule test compounds can initially be members of an organic or inorganic chemical library. As used herein, “small molecules” refers to small organic or inorganic molecules of molecular weight below about 3,000 Daltons. The small molecules can be natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio., 1:60 (1997). In addition, a number of small molecule libraries are commercially available.

The test compound can have a structure that is based on an active fragment of O APN, Ang III, Ang IV, Ang 1-7, Anpep, aminopeptidase A (APA), AT4, Na/K ATPase, aldosterone, Mas and ACE2. For example, computer modeling methods known in the can be used to rationally design a molecule that has a structure similar to an active fragment of APN, Ang III, Ang IV, Ang 1-7, Anpep, aminopeptidase A (APA), AT4, Na/K ATPase, Mas and ACE2 or portions thereof.

In some embodiments, the compounds are optimized to improve their therapeutic index, i.e., increase therapeutic efficacy and/or decrease unwanted side effects. Thus, in some embodiments, the methods described herein include optimizing the test or candidate compound. In some embodiments, the methods include formulating a therapeutic composition including a test or candidate compound (e.g., an optimized compound) and a pharmaceutically acceptable carrier. In some embodiments, the compounds are optimized by derivatization using methods known in the art.

Polynucleotides: In some embodiments, the test compound comprises a polynucleotide that encodes APN, Ang III, Ang IV, Ang 1-7, Anpep, aminopeptidase A (APA), AT4, Na/K ATPase, and ACE2, or an active fragment thereof. In some embodiments, the compound is a polynucleotide that encodes an active fragment of APN, Ang III, Ang IV, Ang 1-7, Anpep, aminopeptidase A (APA), AT4, Na/K ATPase, and ACE2 othat retain ligand binding activity.

Sense Nucleic Acids: In some embodiments, the test compound comprises a polynucleotide that encodes a polypeptide that is at least about 85% identical to the amino acid sequence of APN, Ang III, Ang IV, Ang 1-7, Anpep, aminopeptidase A (APA), AT4, Na/K ATPase, aldosterone and ACE2. In some embodiments, the polynucleotide encodes a polypeptide that is at least about 90%, 95%, 99%, or 100% identical to the full length sequence of APN, Ang III, Ang IV, Ang 1-7, Anpep, aminopeptidase A (APA), AT4, Na/K ATPase, aldosterone, Mas and ACE2 or active fragments thereof. In some embodiments, the active fragment is at least about 10, 12, 14, 16, 18, 20, 22, 24, 26, 28, 30, 32, 34, 36, 38, 39, 40, 45, 50, 55, 60, 65, 70, 75, 80 or more amino acids long. The nucleic acid can include one or more noncoding regions of the coding strand of a nucleotide sequence encoding APN, Ang III, Ang IV, Ang 1-7, Anpep, aminopeptidase A (APA), AT4, Na/K ATPase, aldosterone, Mas and ACE2 (e.g., the 5′ and 3′ untranslated regions). A number of methods are known in the art for obtaining suitable nucleic acids, see, e.g., Sambrook et al., Molecular Cloning: A Laboratory Manual (Cold Spring Harbor Laboratory, Cold Spring Harbor, N.Y.; 3rd ed. 2001).

RNA Interference (RNAi): RNAi is a remarkably efficient process whereby double-stranded RNA (dsRNA, also referred to herein as siRNAs, for small interfering RNAs, or ds siRNAs, for double-stranded small interfering RNAs) induces the sequence-specific degradation of homologous mRNA in animals and plant cells (Hutvagner and Zamore, Curr. Opin. Genet. Dev., 12:225-232 (2002); Sharp, Genes Dev., 15:485-490 (2001)). In mammalian cells, RNAi can be triggered by duplexes of small interfering RNA (siRNA) (Chiu et al., Mol. Cell., 10:549-561 (2002); Elbashir et al., Nature, 411:494-498 (2001)), or by micro-RNAs (miRNA), functional small-hairpin RNA (shRNA), or other dsRNAs which are expressed in vivo using DNA templates with RNA polymerase III promoters (Zeng et al., Mol. Cell, 9:1327-1333 (2002); Paddison et al., Genes Dev., 16:948-958 (2002); Lee et al., Nature Biotechnol., 20:500-505 (2002); Paul et al., Nature Biotechnol., 20:505-508 (2002); Tuschl, T., Nature Biotechnol., 20:440-448 (2002); Yu et al., Proc. Natl. Acad. Sci. USA, 99(9):6047-6052 (2002); McManus et al., RNA, 8:842-850 (2002); Sui et al., Proc. Natl. Acad. Sci. USA, 99(6):5515-5520 (2002)).

The methods described herein can include the use of dsRNA molecules that are targeted to (i.e., bind to) APN, Ang III, Ang IV, Ang 1-7, Anpep, aminopeptidase A (APA), AT4, Na/K ATPase, aldosterone, Mas and ACE2 mRNA.

In a preferred embodiment, the siRNA is directed APN and/or the AT4 receptor. (SEQ ID NOS: 3 and 4).

The dsRNA molecules typically comprise 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is identical or substantially identical to the first strand. Each strand can also have one or more overhanging (i.e., non-complementary) nucleotides, e.g., one, two, three, four or more overhanging nucleotides, e.g., dTdTdT.

The dsRNA molecules can be chemically synthesized, or can be transcribed in vitro from a DNA template, or in vivo from, e.g., shRNA. The dsRNA molecules can be designed using any method known in the art; a number of algorithms are known in the art, see, e.g., Tuschl et al., Genes Dev 13(24):3191-7 (1999), and many are available on the internet, e.g., on the websites of Dharmacon (Lafayette, Colo.) or Ambion (Austin, Tex.).

Negative control siRNAs should have the same nucleotide composition as the selected siRNA, but without significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

micro RNA (miRNAs) of approximately 22 nucleotides can be used to regulate gene expression at the post transcriptional or translational level. miRNAs can be excised in the cell from an approximately 70 nucleotide precursor RNA stem-loop by Dicer, an RNase III-type enzyme, or a homolog thereof. By substituting the stem sequences of the miRNA precursor with miRNA sequence complementary to the target mRNA, a vector construct that expresses the novel miRNA can be used to produce siRNAs to initiate RNAi against specific mRNA targets in mammalian cells (Zeng (2002), supra). When expressed by DNA vectors containing polymerase III promoters, micro-RNA designed hairpins can silence gene expression (McManus (2002), supra).

dsRNA can be delivered directly into cells in vivo or in vitro using methods known in the art, e.g., cationic liposome transfection, nanoparticles, and electroporation, or expressed in vivo or in vitro from recombinant DNA constructs that allow longer-term target gene suppression in cells, including mammalian Pol III promoter systems (e.g., H1 or U6/snRNA promoter systems (Tuschl (2002), supra) capable of expressing functional double-stranded siRNAs; (Bagella et al., J. Cell. Physiol. 177:206-213 (1998); Lee et al. (2002), supra; Miyagishi et al. (2002), supra; Paul et al. (2002), supra; Yu et al. (2002), supra; Sui et al. (2002), supra).

Viral-mediated delivery mechanisms can also be used to induce specific silencing of targeted genes through expression of siRNA, for example, by generating recombinant adenoviruses harboring siRNA under RNA Pol II promoter transcription control (Xia et al. (2002), supra). Transcriptional termination by RNA Pol III occurs at runs of four consecutive T residues in the DNA template, providing a mechanism to end the siRNA transcript at a specific sequence. The dsRNA thus produced is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella et al. (1998), supra; Lee et al. (2002), supra; Miyagishi et al. (2002), supra; Paul et al. (2002), supra; Yu et al. (2002), supra; Sui et al. (2002) supra). Constructs containing siRNA sequence under the control of T7 promoter also make functional siRNAs when cotransfected into cells with a vector expression T7 RNA polymerase (Jacque (2002), supra).

In an animal, whole-embryo electroporation can efficiently deliver synthetic siRNA into post-implantation mouse embryos (Calegari et al., Proc. Natl. Acad. Sci. USA, 99(22): 14236-40 (2002)). In adult mice, efficient delivery of siRNA can be accomplished by “high-pressure” delivery technique, a rapid injection (within 5 seconds) of a large volume of siRNA containing solution into animal via the tail vein (Liu (1999), supra; McCaffrey (2002), supra; Lewis, Nature Genetics 32:107-108 (2002)). Local delivery can also be used, e.g., with a carrier such as lipiodol (iodine in oil) to facilitate delivery into cells.

Engineered RNA precursors, introduced into cells or whole organisms as described herein, can be used for the production of a desired siRNA molecule. Such an siRNA molecule can then associate with endogenous protein components of the RNAi pathway to bind to and target a specific mRNA sequence for cleavage and destruction. In this fashion, the mRNA to be targeted by the siRNA generated from the engineered RNA precursor will be depleted from the cell or organism, leading to a decrease in the concentration of the protein encoded by that mRNA in the cell or organism. Additional information regarding the use of RNAi can be found in RNA Interference Editing, and Modification: Methods and Protocols (Methods in Molecular Biology), Gott, Ed. (Humana Press, 2004);

Antisense Polynucleotides: An “antisense” nucleic acid can include a nucleotide sequence that is complementary to a “sense” nucleic acid encoding a protein, e.g., complementary to all or a portion of the coding strand of a double-stranded cDNA molecule or complementary to APN, Ang III, Ang IV, Ang 1-7, Anpep, aminopeptidase A (APA), AT4, Na/K ATPase, aldosterone, Mas and ACE2, or ligands thereof, mRNA sequence. In another embodiment, the antisense nucleic acid molecule is antisense to a “noncoding region” of the coding strand of a nucleotide sequence encoding APN, Ang III, Ang IV, Ang 1-7, Anpep, aminopeptidase A (APA), AT4, Na/K ATPase, aldosterone, Mas and ACE2, or ligands thereof (e.g., the 5′ and 3′ untranslated regions).

Based upon the sequences disclosed herein, one of skill in the art can easily choose and synthesize any of a number of appropriate antisense molecules for use in accordance with the present invention. For example, a “gene walk” comprising a series of oligonucleotides of 15-30 nucleotides spanning the length of an APN, Ang III, Ang IV, Ang 1-7, Anpep, aminopeptidase A (APA), AT4, Na/K ATPase, aldosterone, Mas and ACE2, or ligands thereof, nucleic acid can be prepared, followed by testing for inhibition of APN, Ang III, Ang IV, Ang 1-7, Anpep, aminopeptidase A (APA), AT4, Na/K ATPase, aldosterone, Mas and ACE2 expression. Optionally, gaps of 5-10 nucleotides can be left between the oligonucleotides to reduce the number of oligonucleotides synthesized and tested. Other methods, including computational analysis, RNAse H mapping, and antisense-oligonucleotide scanning microarrays, can also be used (see, e.g., DNA Microarrays: A Practical Approach, Schena, Ed. (Oxford University Press 1999; Scherr and Rossi, Nuc. Acids Res., 26(22):5079-5085 (1998)).

An antisense nucleic acid can be designed such that it is complementary to the entire coding region of a target APN, Ang III, Ang IV, Ang 1-7, Anpep, aminopeptidase A (APA), AT4, Na/K ATPase, aldosterone, Mas and ACE2 mRNA, but can also be an oligonucleotide that is antisense to only a portion of the coding or noncoding region of the target mRNA. For example, the antisense oligonucleotide can be complementary to a region surrounding the translation start site of the target mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest).

Antisense nucleic acid molecules are typically administered to a subject (e.g., by direct injection at a tissue site), or generated in situ such that they hybridize with or bind to cellular mRNA and/or genomic DNA encoding an APN, Ang III, Ang IV, Ang 1-7, Anpep, aminopeptidase A (APA), AT4, Na/K ATPase, aldosterone, Mas and ACE2 protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter can be used.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al., Nucleic Acids. Res. 15:6625-6641 (1987)). The antisense nucleic acid molecule can also comprise a 2′-o-methylribonucleotide (Inoue et al. Nucleic Acids Res. 15:6131-6148 (1987)) or a chimeric RNA-DNA analogue (Inoue et al. FEBS Lett., 215:327-330 (1987)).

APN, Ang III, Ang IV, Ang 1-7, Anpep, aminopeptidase A (APA), AT4, Na/K ATPase, aldosterone, Mas and ACE2 gene expression can be inhibited by targeting nucleotide sequences complementary to the regulatory regions (e.g., promoter and/or enhancer) to form triple helical structures that prevent transcription of these molecules. The potential sequences that can be targeted for triple helix formation can be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

Ribozymes: Ribozymes are a type of RNA that can be engineered to enzymatically cleave and inactivate other RNA targets in a specific, sequence-dependent fashion. By cleaving the target RNA, ribozymes inhibit translation, thus preventing the expression of the target gene. Ribozymes can be chemically synthesized in the laboratory and structurally modified to increase their stability and catalytic activity using methods known in the art. Alternatively, ribozyme genes can be introduced into cells through gene-delivery mechanisms known in the art. A ribozyme having specificity for an APN, Ang III, Ang IV, Ang 1-7, Anpep, aminopeptidase A (APA), AT4, Na/K ATPase, aldosterone, Mas and ACE2-encoding nucleic acid can include one or more sequences complementary to the nucleotide sequence of an APN, Ang III, Ang IV, Ang 1-7, Anpep, aminopeptidase A (APA), AT4, Na/K ATPase, aldosterone, Mas and ACE2 cDNA, and a sequence having known catalytic sequence responsible for mRNA cleavage (see U.S. Pat. No. 5,093,246 or Haselhoff and Gerlach, Nature, 334:585-591 (1988)). For example, a derivative of a Tetrahymena L- 19 IVS RNA can be constructed in which the nucleotide sequence of the active site is complementary to the nucleotide sequence to be cleaved in an APN, Ang III, Ang IV, Ang 1-7, Anpep, aminopeptidase A (APA), AT4, Na/K ATPase, aldosterone, Mas and ACE2-encoding mRNA. See, e.g., Cech et al., U.S. Pat. No.4,987,071; and Cech et al., U.S. Pat. No. 5,116,742. Alternatively, APN, Ang III, Ang IV, Ang 1-7, Anpep, aminopeptidase A (APA), AT4, Na/K ATPase, aldosterone, Mas and ACE2 mRNA can be used to select a catalytic RNA having a specific ribonuclease activity from a pool of RNA molecules. See, e.g., Bartel, and Szostak, Science, 261:1411-1418 (1993).

Peptides/Polypeptides: In some embodiments, the test compound includes an APN, Ang III, Ang IV, Ang 1-7, Anpep, aminopeptidase A (APA), AT4, Na/K ATPase, aldosterone, Mas and ACE2 polypeptides, or an active fragments thereof. In some embodiments, the compound is an active fragment of APN, Ang III, Ang IV, Ang 1-7, Anpep, aminopeptidase A (APA), AT4, Na/K ATPase, aldosterone, Mas and ACE2 that retains ligand binding activity.

In some embodiments, the test compound comprises a polypeptide that is at least about 85% identical to the amino acid sequence of APN, Ang III, Ang IV, Ang 1-7, Anpep, aminopeptidase A (APA), AT4, Na/K ATPase, aldosterone, Mas and ACE2. In some embodiments, the polypeptide is at least about 90%, 95%, 99%, or 100% identical to the full length sequence of APN, Ang III, Ang IV, Ang 1-7, Anpep, aminopeptidase A (APA), AT4, Na/K ATPase, aldosterone, Mas and ACE2, or an active fragment thereof. In some embodiments, the active fragment is at least about 10, 12, 14, 16, 18, 20, 22, 24, 26, 28, 30, 32, 34, 36, 38, 39, 40, 45, 50, 55, 60, 65, 70, 75, 80 or more amino acids long. A “polypeptide comprising an active fragment of APN, Ang III, Ang IV, Ang 1-7, Anpep, aminopeptidase A (APA), AT4, Na/K ATPase, aldosterone, Mas and ACE2” includes less than the full length of each, but can include other (i.e., non- APN, Ang III, Ang IV, Ang 1-7, Anpep, aminopeptidase A (APA), AT4, Na/K ATPase, aldosterone, Mas and ACE2) proteins or fragments thereof, e.g., fluorescent proteins such as green fluorescent protein (GFP), red fluorescent protein (RFP), blue fluorescent protein (BFP) or yellow fluorescent protein (YFP), or peptides that enhance delivery, e.g., a TAT protein transduction domain (PTD).

Methods of Formulation: The compounds described herein can be incorporated into pharmaceutical compositions. Such compositions typically include the active ingredient and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from pressured container or dispenser which contains a suitable propellant, e.g., a gas such as carbon dioxide, or a nebulizer. Such methods include those described in U.S. Pat. No. 6,468,798. Compositions for inhalation can also include propellants, surfactants, and other additives, e.g., to improve dispersion, flow, and bioavailability.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Compounds comprising nucleic acids can also be administered by transfection or infection using methods known in the art, including but not limited to the methods described in McCaffrey et al. (2002), Nature, 418(6893), 38-9 (hydrodynamic transfection); Xia et al. (2002), Nature Biotechnol., 20(10), 1006-10 (viral-mediated delivery); or Putnam (1996), Am. J. Health Syst. Pharm., 53(2), 151-160, erratum at Am. J. Health Syst. Pharm., 53(3), 325 (1996). Compounds comprising nucleic acids can also be administered by method suitable for administration of DNA vaccines. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88(2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the compounds are prepared with carriers that will protect the active ingredient against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In some embodiments, the compounds (e.g., polypeptides) are modified to enhance delivery into cells, e.g., by the addition of an optimized or native TAT protein transduction domain (PTD), e.g., as described in Ho et al., Cancer Res. 61(2):474-7 (2001). Where the compound is a polypeptide, the polypeptide can be a fusion protein comprising an active portion (e.g., an active fragment of Apoptin) and a TAT PTD fused in frame.

The pharmaceutical compositions can be included in a container, pack, or dispenser together with instructions for administration.

Methods of Treatment: As used herein, the term “treatment” is defined as the application or administration of a therapeutic agent described herein, or identified by a method described herein, to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

Examples of routes of administration include parenteral, e.g., intravenous, intradermal, subcutaneous, oral (e.g., inhalation), transdermal (topical), transmucosal, and rectal administration.

Therapeutic agents include, for example, proteins, nucleic acids, small molecules, peptides, antibodies, siRNAs, ribozymes, and antisense oligonucleotides. Dosage, toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a compound (i.e., an effective dosage) means an amount sufficient to produce a therapeutically (e.g., clinically) desirable result, The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

Kits

The assay of the present invention is also ideally suited for the preparation of a kit. Such a kit may comprise a carrier means being compartmentalized to receive in close confinement there with one or more container means such as vials, tubes and the like, each of said container means comprising the separate elements of the immunoassay. For example, there may be a container means containing a first antibody immobilized on a solid phase support, and a further container means containing a second detectably labeled antibody in solution. Further container means may contain standard solutions comprising serial dilutions of the biomarkers to be detected. The standard solutions of each biomarker may be used to prepare a standard curve with the concentration of each biomarker plotted on the abscissa and the detection signal on the ordinate. The results obtained from a sample containing any one of the may be interpolated from such a plot to give the concentration of each detected biomarker.

In one another embodiment, a kit for diagnosing hypertension in a subject comprising a panel of biomarkers, the kit comprising (a) a substrate for holding a biological sample isolated from a human subject suspected of having salt-sensitive hypertension or salt-resistant hypertension versus salt-independent hypertension and/or hypertensive disorders, (b) a fluorogenic agent that detects at least one or more biomarkers; (c) a panel of biomarkers; and, (d) printed instructions for reacting the agent with the biological sample or a portion of the biological sample to detect the presence or amount of at least one marker in the biological sample. Preferably, the kit comprises a the panel of biomarkers of any one or more of: APN, Ang III, Ang IV, Ang 1-7, Anpep, aminopeptidase A (APA), AT4, Na/K ATPase, aldosterone, Mas and ACE2. Optionally, the kit further comprises antibodies specific for any one or more biomarkers: APN, Ang III, Ang IV, Ang 1-7, Anpep, aminopeptidase A (APA), AT4, Na/K ATPase, aldosterone, Mas and ACE2. Preferably, at least one biomarker in a patient is elevated as compared to a normal healthy control. The kit can provide both the panel of biomarkers and the antibodies if desired.

In an additional embodiment, the invention includes a diagnostic kit for use in screening urine containing antigens of the polypeptide of the invention. The diagnostic kit includes a substantially isolated antibody specifically immunoreactive with polypeptide or polynucleotide antigens, and means for detecting the binding of the polynucleotide or polypeptide antigen to the antibody. In one embodiment, the antibody is attached to a solid support. In a specific embodiment, the antibody may be a monoclonal antibody. The detecting means of the kit may include a second, labeled monoclonal antibody. Alternatively, or in addition, the detecting means may include a labeled, competing antigen.

In one diagnostic configuration, test serum is reacted with a solid phase reagent having a surface-bound antigen obtained by the methods of the present invention. After binding with specific antigen antibody to the reagent and removing unbound serum components by washing, the reagent is reacted with reporter-labeled anti-human antibody to bind reporter to the reagent in proportion to the amount of bound anti-antigen antibody on the solid support. The reagent is again washed to remove unbound labeled antibody, and the amount of reporter associated with the reagent is determined. Typically, the reporter is an enzyme which is detected by incubating the solid phase in the presence of a suitable fluorometric, luminescent or colorimetric substrate (Sigma, St. Louis, Mo.).

The solid surface reagent in the above assay is prepared by known techniques for attaching protein material to solid support material, such as polymeric beads, dip sticks, 96-well plate or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

Optionally, the kit may further comprise a standard or control information so that the test sample can be compared with the control information standard to determine if the test amount of a marker detected in a sample is a diagnostic amount consistent with a diagnosis of salt-sensitive hypertensive versus salt-resistant hypertensive versus salt-independent hypertensive and/or hypertensive disorders and/or effect of treatment on the patient, and/or effective dosing of a therapeutic molecule.

In another embodiment, a kit comprises: (a) a substrate comprising an adsorbent thereon, wherein the adsorbent is suitable for binding a marker, and (b) instructions to detect the marker or markers by contacting a sample with the adsorbent and detecting the marker or markers retained by the adsorbent. In some embodiments, the kit may comprise an eluant (as an alternative or in combination with instructions) or instructions for making an eluant, wherein the combination of the adsorbent and the eluant allows detection of the markers using gas phase ion spectrometry. Such kits can be prepared from the materials described above, and the previous discussion of these materials (e.g., probe substrates, adsorbents, washing solutions, etc.) is fully applicable to this section and will not be repeated.

In another embodiment, the kit may comprise a first substrate comprising an adsorbent thereon (e.g., a particle functionalized with an adsorbent) and a second substrate onto which the first substrate can be positioned to form a probe which is removably insertable into a gas phase ion spectrometer. In other embodiments, the kit may comprise a single substrate which is in the form of a removably insertable probe with adsorbents on the substrate. In yet another embodiment, the kit may further comprise a pre-fractionation spin column (e.g., Cibacron blue agarose column, anti-HSA agarose column, size exclusion column, Q-anion exchange spin column, single stranded DNA column, lectin column, etc.).

Optionally, the kit can further comprise instructions for suitable operational parameters in the form of a label or a separate insert. For example, the kit may have standard instructions informing a consumer how to wash the probe after a sample is contacted on the probe. In another example, the kit may have instructions for pre-fractionating a sample to reduce complexity of proteins in the sample. In another example, the kit may have instructions for automating the fractionation or other processes.

Without further elaboration, it is believed that one skilled in the art can, using the preceding description, utilize the present invention to its fullest extent. The invention will be further clarified by a consideration of the following examples, which are intended to be purely exemplary of the present invention are thus to be construed as merely illustrative examples and not limitations of the scope of the present invention in any way.

### EXAMPLES

Materials and Methods

All of the techniques required are currently being or have recently been performed by our laboratory. All reagents are commercially available, including Abs to Anpep, ACE2, APA, and Mas for Western analysis and immunohistochemistry. HK2 and LLCPK1 cells are from ATCC.

Male Dahl SS/Jr, Dahl SR/Jr, SHR, Lewis, and Sprague Dawley rats are purchased from Harlan Sprague Dawley (Indianapolis, Ind.). Rats are maintained on Purina 5501 chow containing 8% (high) or 0.3% (low) NaCl (water ad lib). Since hypertension is also linked to obesity and rats on a high salt-diet are inclined to a higher caloric intake, rats are restricted to 20 g and 5 g respectively of chow per day and weights monitored.

Continuous arterial blood pressure monitoring is by radiotelemetry in freely moving instrumented rats. An implantable osmotic pump is used for infusion experiments.

Ang II metabolites are analyzed using an LCMS that has been developed. This represents a significant advance over HPLC, which has been used alone to measure Ang metabolites in plasma and kidneys (Campbell D. J., et al. Hypertension. 1991;18:763-773). The assay is highly specific and can quantitate each Ang II metabolite simultaneously. The sensitivity of this assay is 0.1 pg (SN=3). In pilot studies, Ang IV in LLCPK cell extracts has been on the order of 600 pg/10⁵ cells and in the cell culture media of 125 pg/ml indicating formation of endogenous Ang IV by the cells. These measurements are complemented with QIHC with specific Abs that we have developed to Ang II metabolites. Urine, plasma, and renal tissue angiotensins are extracted in acid ethanol with peptidase inhibitors.

For quantitative immunohistochemistry, a standard three-stage indirect immunoperoxidase technique is used on formalin fixed kidney sections. Images are acquired using a 4.9 million pixel digital scanning 24-bit RGB camera, saved in TIFF format, and used for subsequent analysis. QIHC is performed after digitally dissecting out the relevant portion of the image for analysis, and processing using a program (Ricci A, et al. J. Histochem Cytochem. 2002;50: 1091-1096; Siegsmund M, et al. J Am Soc Nephrol. 2002; 13:1847-1854; also available at www.uic.edu/com/dom/gastro/Freedownloads.html).

Anpep, APA, and ACE2 activity is measured using fluorogenic peptides. ²²Na flux is measured in cultured cells using an Ussing Chamber.

Na/K ATPase expression and activity are measured by basolateral surface biotinylation and oubain-inhibitable ATPase activity, respectively, in cells cultured in Transwell plates.

Urine Assay Protocol

This assay was done in triplicates with 1:5 dilution and 1:10 dilution. The following table indicates how the dilutions were made:

| | Urine (μl) | Water (μl) | 2xPBS (μl) | Total (ml) |
|---|---|---|---|---|
| A (1:5) | 200 | 800 | 1000 | 2 ml |
| B (1:5) | 200 | 800 | 1000 | 2 ml |
| C (1:5) | 800 | 3200 | 1000 | 2 ml (after doing |
| | | | | the 1:10 |
| | | | | dilutions). |
| D (1:10) | 1000 from C | 1000 | 1000 | 2 ml (1000 ul |
| | | | | thrown out after |
| | | | | mixing) |
| E (1:10) | 1000 from C | 1000 | 1000 | 2 ml (1000 ul |
| | | | | thrown out after |
| | | | | mixing) |
| F (1:10) | 1000 from C | 1000 | 1000 | 2 ml (1000 ul |
| | | | | thrown out after |
| | | | | mixing) |

After the urine dilutions were done, a reading was taken on the spectrophotometer at 405 nm. This was the background value. The concentration of substrate (alanine-p-nitroanilide) used was 0.0147 mg/ml. 150 μl of substrate was added to each tube. The solution was allowed to incubate at 37° C. for an hour. A second reading was taken at 405 nm in the spectrophotometer. The background from this reading was subtracted to give the true absorbance value. Note: For cell extracts, instead of urine the extract was added and made up the volume with IX PBS.

APN Standard Curve

| | |
|---|---|
| Std 1 (0.714 ug/ml): | 1 ml 1XPBS + 1 ul APN (recombinant |
| | protein) + 1 ml 2X PBS |
| Std 2 (0.143 ug/ml): | 400 ul of Std. 1 + 1600 ul H₂O + 1 ml 2X PBS |
| Std 3 (0.0714 ug/ml): | 1000 ul of Std. 2 + 1000 ul H₂O + |
| | 1 ml 2X PBS (throw 600 ul) |
| Std 4 (0.0143 ug/ml): | 400 ul of Std. 3 + 1600 ul H₂O + 1 ml 2X PBS |
| Std 5 90.00714 ug/ml): | 1000 ul of Std 4 + 1000 ul H₂O + |
| | 1 ml 2X PBS (throw 600 ul) |
| Std 6 (0.00143 ug/ml): | 400 ul of Std. 5 + 1600 ul H₂O + |
| | 1 ml 2X PBS (throw 1000 ul) |

### Example 1

### Renal Angiotensin II Metabolites and Expression of Peptidases in Salt-resistant and Salt-sensitive (SS) Strains of Rat on Low and High Salt Diets

Without wishing to be bound by theory, the overall hypothesis is that Ang II metabolites regulate adaptation to high salt and that urinary angiotensin metabolites can be used to classify salt-sensitive and salt-resistant forms of hypertension.

We have shown that Anpep expression/activity (Farjah M, Roxas B, and Danziger, R. S. Hypertension 43(2), 282-285. 2004) and have results that Ang IV levels are greater in kidneys and renal tubules from normotensive and salt-resistant rat strains on 8% versus 0.3% NaCl diets. This tests the hypothesis that Ang II metabolism plays a mechanistic role in adaptation to high salt and that it contributes to salt-sensitivity in the Dahl SS rat. The first objective is to measure renal tubule Ang III, Ang IV and Ang 1-7 levels in salt-sensitive (Dahl salt-sensitive) and salt-resistant (Dahl salt-resistant (SR), Sprague Dawley, and Lewis) rat strains on 0.3 and 8% NaCl diets. These are performed using 1) a powerful technology, i.e., liquid chromatography—mass spectroscopy (LCMS), which permits the simultaneous characterization and measurement of all Ang II metabolites, and 2) quantitative immunohistochemistry (QIHC) with specific monoclonal/polyclonal Abs that we have developed to Ang II metabolites to measure expression in proximal and distal tubule segments specifically. Using this technique, we will also measure Ang 1-9, Ang 2-10, Ang 1-5 and Ang 3-7. If significant differences in these are found, these will be followed-up upon. In parallel studies, the 1) expression/activity of peptidases that form the peptides, i.e. APA for Ang III, Anpep for Ang IV, and ACE2 for Ang 1-7, and 2) expression of the Angiotensin IV (AGTRIV)3 and Angiotensin 1-7 receptor (Mas)4 are measured. Salt-resistant strains should have greater Ang IV (and Anpep) and Ang 1-7 (and ACE2), and less Ang II and Ang III (and APA) versus Dahl SS rats, consistent with a reduction in Na uptake mediated by a coordinated action of Ang II metabolites with specific actions.

In additional studies to more critically reveal regulation of salt-adaptation by Ang II metabolism in kidneys and link the activity of established peptidases to formation of specific Ang II metabolites in vivo, the effect of infusion of Anpep, APA, and ACE2 inhibitors, e.g., bestatin, EC33.RB150, and MLN4760, respectively, on Ang metabolites and salt-sensitivity are determined. In preliminary experiments, bestatin, an inhibitor of Anpep activity, induced sensitivity in Dahl SR rats. For these experiments, continuous radiotelemetric blood pressure monitoring is performed in rats instrumented with transducers and osmotic mini-pumps.

The second objective is to investigate regulation of Na+flux and basolateral activity/expression of Na/K ATPase, the principal Na transporter in renal tubules, in proximal tubule suspensions and cultured human and rat kidney epithelial cells, HK2 and NRK cells respectively, by specific Ang II metabolites. Signaling by metabolites with the greatest difference in abundance in salt-resistant vs. salt-sensitive rats are reasoned to most likely to play a physiological role in salt-adaptation and are focused upon in these studies. Angiotensin II metabolites Ang III, Ang IV, Ang 1-7 should be among these. It is expected that the results from these sets of experiments will identify the most important Ang II metabolites in salt-adaptation.

### Example 2

### Ang II Metabolites and Relevant Amino/carboxypeptidases in the Urine and Plasma of Salt-resistant, Salt-sensitive, and Salt-independent Hypertensive Rat Strains

This determines the relationship between urinary and renal Ang II metabolites. In the proposed experiments, urinary and plasma Ang III, Ang IV, Ang 1-7, Anpep, Aminopeptidase A (APA), and ACE2 are measured in salt-resistant, i.e., Dahl SS, Dahl SR, Sprague-Dawley, and spontaneously hypertensive (SHR) rats on low and high salt diets. These are related to indexes of renal function, including creatinine clearance. It is expected that the results will show that renal Ang II metabolism is reflected in the urine. The results of the proposed experiments will provide both 1) a comprehensive analysis of Ang II metabolism in salt-adaptation and salt-sensitive hypertension and 2) a focus for subsequent studies directed at discovering the physiologically relevant Ang II metabolites. This would lead to the development of novel therapeutic targets/agents and classification system for hypertension and/or salt-sensitivity based on urinary/plasma Ang metabolites.

While multiple signaling pathways, e.g., nitric oxide, natriuretic peptides, endothelin, renal, endocrine, and vascular mechanisms, all have been proposed to conspire in complex ways to produce the salt-sensitive hypertensive phenotype, an inability to increase Na excretion is central to each.

It is generally accepted that the RAS plays a central role in the regulation of renal Na excretion and blood pressure. The classical RAS pathway is defined by the metabolism of angiotensinogen by renin to Ang I and of Ang I to Ang II by angiotensin converting enzyme (ACE). Ang II activates AT1 and AT2 receptors. However, there is evolving evidence that metabolites of Ang II may also be important in RAS and are differentially regulated. Specifically:

Ang III 2-8 (R-V-Y-I-H-P-F (SEQ ID NO: 1)) is formed by hydrolysis of Ang II by Aminopeptidase A (APA; glutamyl aminopeptidase EC 3.4.11.7), which is highly expressed in the kidney. Ang III is generally considered equipotent to Ang II with regard to effects on blood pressure, aldosterone secretion and renal functions. It has been suggested that it activates AT2 preferentially to AT1 in the kidney. The metabolic clearance rate of Ang III is five times that of Ang II. In our results with primary Ab that we developed to Ang III, Ang III signal was detected in renal tubules and vasculature.

Ang IV 3-8 (V-Y-I-H-P-F (SEQ ID NO: 2)) is formed from Ang III by renal tubule Anpep. Ang IV decreases transcellular Na+transport (as measured by proximal tubule O₂ consumption rates) in fresh suspensions of control or nystatin-stimulated (bypasses rate-limiting step of apical Na+entry) rat proximal tubules. It increases Na uptake (reflecting decreased flux) in HK-2 cells. Ang IV infusion into the renal artery increases urinary Na excretion; increases cortical blood flow; and lowers blood pressure. We have linked Anpep is adaptation to high salt and discovered that Anpep inhibits basolateral Na/K ATPase, the principal Na transporter in tubules, via Ang IV signaling.

The receptor of Ang IV, AT4, has been identified as insulin-regulated membrane aminopeptidase (IRAP). AT4 receptors have been detected in human PTs, DT/collecting duct, vascular smooth muscle, and endothelial cells.

Ang 1-7 is formed directly from Ang II and from Ang I by hydrolysis to Ang 1-9 with subsequent metabolism by ACE and neprilysin hydrolysis, and by ACE2, a captopril insensitive carboxypeptidase homologue of ACE, respectively. Ang 1-7 is expressed in the arterial vasculature and myocardium. Ang 1-7 is reported, as opposed to Ang II and III, to vasodilate and have antiproliferative actions. Vascular actions of Ang 1-7 have been traced to inhibition of Ang II effects; increased production of nitric oxide, vasodilatory prostanoids, and endothelium-derived relaxing factor; and potentiation of bradykinin effects.

An endogenous G-protein coupled receptor (Mas) in heart and blood vessels (mRNA detected in testis, kidney, and brain as well) for Ang 1-7 has been identified by Santos et al. (Proc Natl Acad Sci U S A. 2003;100:8258-8263).

Others: Ang (1-9); Ang (1-5); Ang (3-5); Ang (2-7); Ang (3-7); Ang (2-10) (see FIG. 10) are formed by ACE and aminopeptidases, however, they have unknown functions although they have been detected in kidneys and plasma. If any of these is found to be present in significant concentrations and/or have significantly different abundance in salt-sensitive vs salt-resistant rat strains, we will determine their effect on vascular tone and renal tubule cell Na transport.

This is a first stage of our long-term goal of developing individualized and innovative therapeutic approaches to hypertension based on physiology and genetics.

The results of this work will serve as a foundation for our future studies directed at 1) evaluating Angiotensin II metabolism and metabolites in human forms of hypertension and 2) determining the utility of urinary and/or plasma Ang II metabolites as biomarkers for the identification and, even perhaps, classification of hypertensives, and 3) developing novel therapeutic targets/agents.

### Example 3

### To Define Regulation of Renal APN, Ang IV, Ang III, and AT4 by NaCl

Preliminary data indicates that renal APN abundance and activity is greater in Dahl SR/Jr and normotensive rat strains (but not the Dahl SS/Jr rat) on 8% versus 0.3% NaCl. This should determine that renal adaptation to high salt is associated with increased APN and Ang IV in renal tubule cells. If an increase in renal tubule APN plays a mechanistic role in salt-adaptation, salt-resistant rat strains should have greater APN abundance in salt-resistant rats on high versus low NaCl. If Ang IV abundance in renal tubules is specifically linked to formation by APN, then transgenic mice in which APN is over-expressed should have increased Ang IV. That APN abundance in rats on high NaCl is less in the Dahl SS/Jr rat than in normotensive strains on high salt may be indicative of regulation of APN by blood pressure since the Dahl SS/Jr rat is hypertensive on high NaCl. If this is the case, renal APN abundance should also be less in the spontaneously hypertensive rat (SHR) than in normotensive strains.

Experimental Approach: Rats and mice are phenotyped for blood pressure and salt-sensitivity by continuous radiotelemetric monitoring of arterial blood pressure on 8% and 0.3% NaCl diets (for 10 days each). Expression of Ang III, Ang IV, APN, and AT4 receptors in specific cells, PT, DT, and CCDs are measured using quantitative immunohistochemistry.

Experiments: Renal tubule APN, Ang IV, and AT4 increase in normal adaptation to high salt diet. Renal tubule APN, Ang IV, Ang III, AT4 abundance and basolateral Na/K ATPase expression/activity are measured in normotensive (Sprague Dawley) and salt-resistant (Dahl SR/Jr) rat strains (all male, 250-300 g) on 0.3% and 8% NaCl diets.

Reduced renal tubule APN, Ang IV, and AT4 in the Dahl salt-sensitive (SS/Jr) and/or spontaneously hypertensive (SHR) strains. Renal tubule APN, Ang IV, Ang III, and AT4 abundance is measured in Dahl SS/Jr rats on 0.3% and 8% NaCl diet.

Increased intracellularAng IV linked to increased APN in renal tubules in the intact kidney. Ang IV and the AT4 receptor are measured in kidneys from transgenic mice overexpressing the APN gene. Studies are planned for transgenic mice with APN over-expression.

Animals: Rats/diets: Male Dahl SS/Jr, Dahl SR/Jr, SHR, Lewis, and Sprague Dawley rats from Harlan Sprague Dawley (Indianapolis, IN). Mice: Transgenic mice for the human APN gene (transgene engineered to use proximal human APN promoter, active in epithelial cells of the intestine, lung, liver, and kidney) and non-transgenic, age/sex matched FVB/NJ controls are used. Male mice (40-50 g) are used for these studies. Only males are studied in this proposal to eliminate estrus cycle as a confounder. Studying potential gender differences is of great interest and are incorporated in the studies at a later stage. The salt is administered in chow from the 5501 series from Purina containing 8% (high) or 0.3% (low) NaCl and given water ad lib (same diets for rats and mice) x 10 days. Since hypertension is also linked to obesity and rats on a high salt-diet are inclined to a higher caloric intake, rats and mice are restricted to 20 g and 5 g respectively of chow per day and weighed. Blood pressures are measured to determine salt-resistance and sensitivity. Urinary Na is measured and creatinine clearance calculated.

APN, Ang IV, Ang III, AT4, Na/KATPase measurements: Quantitative immunohistochemistry (QIHC) is used so that the analysis of protein expression in specific segments and organelles of kidneys may be made simultaneously in intact kidney sections.

Immunohistochemistry: A standard three-stage indirect immunoperoxidase technique is used. Briefly, fixed tissue sections are rehydrated in graded alcohols and then rinsed in a running water bath. To quench endogenous peroxidase activity, slides are preincubated in 3% hydrogen peroxide in a light impermeable chamber. After they are washed in PBS, slides are incubated in blocking solution [5% skim milk (vol/vol) and 0.15% H₂O₂ (vol/vol) in deionized water]. The primary antibody for immunohistochemistry for Ang IV and Ang III are developed in rabbits using KLH-conjugated Ang IV peptide. Specificity was confirmed by ELISA to Ang IV and III (titers 50:12800 and 50:6400). Polyclonal anti-human aminopeptidase N antibody (Santa Cruz Biotech, CA); polyclonal Ab to AT4 (Alpha Diagnostics, San Antonio) and monoclonal Ab to the alpha subunit of Na/K ATPase (Upstate, New York) are used. CCD cells are identified by HV-ATPase. After slides are washed again in PBS, primary antibodies to aminopeptidase N and Ang IV are applied, and the tissues incubated for 1 h in a humidity chamber (control tissues are processed similarly except that primary antibodies are not applied). After being washed again in PBS, the tissues are incubated with biotinylated anti-rabbit IgG (DAKO) for 15 min. After they are washed in PBS, the slides are incubated with streptavidin conjugated to horseradish peroxidase (DAKO) for 15 min and washed again in PBS buffer. Incubating slides with the liquid DAB substrate-chromogen system (DAKO) for 5 min identified bound antibody. After a final wash in PBS and distilled water, the slides are counterstained with either Gill's or Harris' modified hematoxylin for 4 min, dehydrated in graded alcohols, and mounted with a cover slip using Permount.

Quantitation: Quantitative immunohistochemistry (QIHC), while subject to changes associated with fixing tissue, has proven to be an extremely useful technique for measuring the abundance of proteins in specific cells within tissues. It provides the great advantage of simultaneously measuring the abundance and distribution of specific proteins in precise cells and organelles, which is of particular value in the proposed studies where the distribution and abundance of specific proteins are compared between tissues.

Measurement by Western analysis of APN, Ang IV, Ang III, AT4, and Na/K ATPase protein in proximal tubule preparations are performed in parallel and complementary experiments. Abs described above are used. We are also exploring the use of ELISAs to measure APN; AT4; and, based on the specific Abs that we have developed, Ang III and Ang IV. Na/K ATPase activity is also measured in PTs isolated from the kidney

Images are acquired using a 4.9 million pixel digital scanning g 24-bit RGB camera, saved in TIFF format, and used for subsequent analysis. QIHC is performed after digitally dissecting out the relevant portion of the image for analysis, and processing using a program written in C (available at uic.edu/com/dom/gastro/Freedownloads.html). This algorithm involves subtracting the energy of the digital file encoding the control image (i.e., not exposed to antibody) from that of the experimental image (i.e., antibody-treated). In this manner, the absolute amount of antibody-specific chromogen per pixel can be determined for any cellular region or structure.

Blood pressure measurements: Radiotelemetry is the state-of-the-art technique for phenotyping blood pressure in rats and possesses several major advantages over tail-cuff syphgmomanometry which is the conventional approach used in most previously published studies. First, for tail-cuff measurements, the rats must be restrained which falsely elevates the blood pressure due to adrenergic storm. With radiotelemetry, the rats are unrestrained and blood pressures are measured continuously in freely moving rats in large cages. Second, tail cuff estimates only systolic pressure while radiotelemetry measures both systolic and diastolic pressures. Third, with tail cuff, blood pressures are measured once or at most twice a day, whereas, with radiotelemetry, blood pressures are measured continuously, allowing assessment of diurnal patterns and the time-integral of overall blood pressure burden. Fourth, with tail cuff the measurements are indirect and subject to all the problems (and more) with indirect sphygmomanometry in humans. In contrast, telemetry provides direct measurements of arterial pressure.

Survival Surgery and Radiotelemetry Measurement of Central Aortic Pressure. Using sterile technique, a radio-transmitter (TA11PA-C20, Data Sciences Intl. for rat or PA-C10 for mice) catheter is inserted via a midline abdominal incision into the left femoral artery and sutured after the tip has been advanced into the proximal aorta. The catheter is tunneled under the skin and the body of the transmitter positioned in a subcutaneous pocket near the right flank of the rat. Xylocaine Jelly (2%) will be pasted around the incision and ampicillin (100 mg/day i.m.) is injected for the first 2-3 postoperative days. Each rat is housed individually in a dedicated room in the Animal Care Facility. Data acquisition will begin 7 days after surgery when normal diurnal blood pressure variability is observed. The data is acquired, processed, and stored using a customized A-D board and Dataquest software (Data Sciences International, Inc.). Once the transmitter is activated by a special magnet, measurements of pulsatile central aortic blood pressure is recorded continuously by placing an individual receiver under each rat's cage. Heart rate and systolic blood pressure, derived from the blood pressure wave form, are averaged during three one hour intervals on each of three days while the rats are resting quietly.

Statistics: For quantitative immunohistochemistry, signals are compared between strains and diets using ANOVA and paired t-tests. Telemetric blood pressure measurements on the two salt diets are compared by ANOVA (to reduce variability, measurements during light and dark cycles will be analyzed separately). Significance P<0.05.

Results obtained will show that renal adaptation to high salt is associated with increased APN and Ang IV in AT4 receptor containing cells.

Blood pressures: Dahl SR/Jr will be normotensive on 0.3% and 8% NaCl diets. Dahl SS/Jr rats will be hypertensive on 8% but not 0.3% NaCl diets (while predictable, confirmation of these phenotypes is important due to genetic drift and “fixation” of genes in strains over time). Sprague Dawley and Lewis rats will have slightly higher blood pressures (but not at hypertensive levels) on 8% versus 0.3% NaCl diets. Transgenic mice with over-expression of APN will be normotensive and salt-resistant. Transgenic mice will be salt-resistant, as will be the controls (Note: the primary rationale for using the transgenic mice is to further associate APN to Ang IV).

APN, Ang IV, Ang III, AT4, and basolateral Na/K ATPase expression/activity measurements: 1. Normotensive, including Dahl SR/Jr, rats on 8% versus 0.3% NaCl will exhibit greater abundance of APN, Ang IV, and AT4 receptors, and lesser abundance of Ang III, in the proximal and CCDs. 2. Dahl SS/Jr rats will have less abundance of APN, Ang IV, and AT4 in both proximal and collecting ducts than normotensive strains. 3. SHR rats will have the same renal abundance and distribution of APN, Ang IV, and AT4 receptors in tubules as in normotensive rats and Dahl SR/Jr rats on the same NaCl diet. 4. Transgenic mice over-expressing APN will have lower renal Ang III and greater Ang IV levels than controls. 5. Salt-resistant rats, e.g., Dahl SR, on 8% versus 0.3% NaCl will have less basolateral Na/K ATPase expression in PT/ DTs and less activity in isolated PT cells.

Data Analysis: Salt-sensitivity and blood pressure data will be integrated with APN, Ang IV, Ang III, and AT4 measurements. Rat strains will be classified as salt adaptive or salt-sensitive based on their mean arterial blood pressure on 8% vs 0.3% NaCl. If there is a significant increase in blood pressure, rats will be categorized as salt-sensitive. Otherwise, they will be classified as “salt-resistant.” Rats: If, APN content is greater in salt-resistant strains, on 8% versus 0.3% NaCl, this suggests that an increase APN is associated with salt-adaptation. If it is increased in only PT cells, we will conclude that it does not mediate any adaptive responses in the CCDs. If APN is not greater in salt-resistant strains on 8% versus 0.3% NaCl diets, this indicates that APN does not mediate salt-resistance in these strains. If APN is lower in SHR rats and Dahl SS/Jr rats on 8% NaCl than in normotensive strains, this suggests that reduced APN can also cause hypertension and we will analyze APN expression in other hypertensive strains, e.g., Milan hypertensive and spontaneously hypertensive—stroke prone (SHR-SP) rats to further test this. If PT and collecting duct APN is greater in transgenic mice over-expressing APN, this suggests both that APN increases Ang IV. If parallel changes in APN are not observed in PT, DT, and CCD, this suggests that APN signaling is not important in salt-adaptation throughout the tubules. If Ang IV abundance in tubule cells is greater in salt-resistant strains on 8% versus 0.3% NaCl, this is consistent with Ang IV-mediated regulation of Na/K ATPase which plays a mechanistic role in salt-adaptation. If Ang IV is not greater in these strains, this suggests that 1) Ang IV is not involved in salt-adaptation and 2) any link between APN increased APN and salt-adaptation is not mediated by Ang IV. If the relationship between APN with Ang IV abundance is the same in the different strains and in the same rat strains on 8% and 0.3% NaCl, i.e., greater APN associated with greater Ang IV, this is supportive of our hypothesis that APN controls Ang IV. If there is a discrepancy between APN and Ang IV abundance changes, this suggests that 1) other enzymes are producing Ang IV; 2) metabolism of Ang IV is altered or 3) that substrate, e.g. Ang III, are limiting. The latter possibility is addressed by Ang III measurements. The first two possibilities are addressed above.

If AT4 abundance is different in salt-resistant rats on 8% versus 0.3% NaCl, this suggests that AT4 may also play a mechanistic role in Ang IV signaling in salt-adaptation If AT4 receptor is not different, this suggests that AT4 expression does not play a role in salt adaptation. If renal tubule Na/K ATPase expression and activity are less in salt-resistant rats on 8% versus 0.3% NaCl, this is consistent with a reduction in the expression of basolateral Na/K ATPase is a major renal mechanism for adaptation to high salt. If this is not observed in a particular salt-resistant strain, this suggests that other transporters/channels may be playing a principal role in salt-adaptation in the strain.

Transgenic mice: If these mice have increased APN expression in tubules, they will provide complementary data to that from the rats. If they are salt-resistant, this is consistent with but does not prove that APN is linked to salt-adaptation unless controls are salt sensitive. If Ang IV abundance is greater in tubule cells where APN is over-expressed when mice are on a 0.3% NaCl diet, this provides good evidence of a link between APN and Ang IV. If Ang IV abundance is not greater, this indicates APN does not increase Ang IV and that signaling for APN is not through Ang IV in the mouse. From these experiments, we expect showing that normal adaptation to high salt diet is associated with an increase in APN in PT and distal nephrons and, its product Ang IV.

Regulation of Na/KATPase in renal epithelial cells by APN: These experiments are conducted to link APN, Ang III and Ang IV to regulation of basolateral Na/K ATPase expression. Without wishing to be bound by theory, increased APN mediates normal adaptation to high salt. We will assess whether APN is linked to reduction of basolateral Na/K ATPase expression, presumably through endocytosis. The mechanism may be through the natriuretic peptide Ang IV, which is formed from Ang III, by APN and activates P38 MAPK and Erk-1, both of which stimulate endocytosis. If an increase in tubule APN plays a mechanistic role in renal adaptation to high NaCl, then APN should inhibit basolateral Na/K ATPase. Since Ang IV is formed by APN and has been shown to both inhibit Na uptake and activate kinases, i.e., P38 MAPK and Erk-1, that have been shown to mediate endocytosis, Ang IV may 1) mediate APN inhibition of Na/K ATPase and 2) reduce basolateral Na/K ATPase expression by endocytosis of the transporter, a well established mechanism for its regulation.

Our data show that Ang IV reduces blood pressure in hypertensive Dahl SS rat, i.e., rats on 8% NaCl diet. If reduced signaling by Ang IV plays a physiological role in adaptation to high salt by reducing basolateral Na/K ATPase activity, then Ang IV should reduce Na/K ATPase in proximal tubule cells isolated from Dahl SR on a normal salt-diet to a greater extent than that in rats on a high salt diet. If APN/Ang IV signaling are reduced in the Dahl SS rat, then Ang IV should reduce Na/K ATPase activity in rats on normal and high diets if signaling by the AT4 receptor is intact.

Experimental Approach: -Cultured epithelial cells: Cell cultures include cultured proximal tubule renal epithelial cell lines, i.e., LLC-PK1 cell in which preliminary studies have been performed, and human kidney (HK2) cells for applicability to humans. Regulation of Na/K ATPase expression by APN is evaluated by inhibiting endogenous APN with siRNA and stimulating APN activity with over-expression of recombinant APN. As a control, the effect of recombinant APN on Na/K distribution in cells treated with siRNA to APN is evaluated. Basolateral Na/K ATPase expression and endocytosis are measured by surface biotinylation and confocal microscopy. Na/K ATPase activity is also measured.

Regulation of Na/K ATPase expression by Ang IV is tested by treating cells with Ang IV and divalinal Ang IV, an AT4 binding peptide generally considered to be a partial inhibitor of the receptor. As control, cells are treated with Ang III.

The role of Ang IV signaling in APN mediated regulation of Na/K ATPase is determined using siRNA to the AT4 receptor. The effect of siRNA to the AT4 receptor on the 1) increase in Na/K ATPase expression and activity caused by siRNA to APN and 2) decrease in Na/K ATPase caused by APN over-expression are examined. As a control, the effect of exogenous Ang IV on Na/K ATPase expression are measured in cells treated with siRNA to the AT4 receptor.

The role of APN in conversion of Ang III to Ang IV is evaluated by determining the impact of treating cells with Ang III (10⁻⁷-10⁻⁹ M) in the presence and absence of siRNA to APN to inhibit APN expression and bestatin to inhibit catalytic activity. Since Ang III activates AT1 receptors, which may antagonize Ang IV-mediated reductions in Na/K ATPase expression, cells are also treated with an AT1 receptor blocker to eliminate this possibility.

The role of P38 MAPK and Erk-1/2 in regulation of basolateral Na/K ATPase by APN is evaluated by determining the impact of 1) p38MAPK inhibitors SB 203580 and Erk activation (MEK) inhibitor PD 169316 on APN over-expression-induced Na/K ATPase redistribution and 2) siRNA to APN and APN over-expression on P38 MAPK and Erk-1 activation. As a control, and to more closely link reduced expression of Na/K ATPase to the kinases, activated forms of these kinases are expressed in the cells. If neither inhibitor completely prevents APN mediated regulation of Na/K ATPase expression, both are used simultaneously to address the possibility the Erk and P38 MAPK act in parallel, as opposed to sequential, pathways in regulating endocytosis in these cells. Erk-2 may suppress APN expression in endothelial cells, so APN expression is to be measured in renal epithelial cells in which Erk signaling is inhibited or activated Erk is over-expressed.

Dahl SS and SR rats: Na/K ATPase activity is measured in proximal tubule cells prepared from rats on 0.3% and 8% NaCl diets and incubated with Ang IV.

Methods: Cells: Renal epithelial cell lines, i.e., LLC-PK and HK2, cells from ATCC are used for these experiments. Cells are grown in high-glucose DMEM media in 75-cm2 flasks. Cells of passage 25 to 40 are used and plated on semipermeable plates (Transwell) at a density of 2×10⁴ cells/ml. Cells are used 10 days after plating and fed fresh incubation medium on alternate days. For immunohistochemical experiments, cells are grown on 4 well chamber slides. Proximal tubules cell are prepared by the method of Periysamy et al. Kidney Int. 2005; 67:1868-1877.

Recombinant cells: Cells are transfected (Lipofectamine 2000 (Qiagen Inc)) with APN cDNA cloned in inducible pcDNA3. 1-Neo vector harboring APN cDNA and stable cell selected by neomycin resistance. Expression is confirmed by Western Analysis. APN induction is by tetracycline.

Measurement of Na/KATPase expression and activity: Na/K ATPase activity in membranes isolated from proximal tubule fractions and membranes from cultured/transfected cell lines is measured as the rate of inorganic phosphate, using coloring reagent (10% ammonium molybdate in 10 N sulfuric acid +ferrous sulfate) released in the presence or absence of oubain.

In complementary experiments, basolateral Na/K ATPase expression in cultured cells is measured both by biotinylation and confocal microscopy. Biotinylation technique to assess the surface Na/K ATPase protein has already been established in our laboratory. Biotinylation is performed as using NHS-S-S-biotin (Pierce). Labeling for 20 min at the basolateral domain in cells grown in transwells at 4° C. to stop endocytosis and internalization of antigen. Biotinylated antigens are immunoprecipitated with streptavidin agarose and released by incubation in 10 mM dithiothreitol, reconstituted in Laemmli buffer, subjected to SDS-PAGE and then probed with Anti alphal Na/K ATPase mAb (Upstate/ Millipore, Billerica, Mass.). For analysis, surface Na/K ATPase is compared to total protein as determined by immunoblotting in solubilized cell extract and with the amount of Na/K ATPase not removed by avidin precipitation method (intracellular pool).

Confocal microscopy: Confocal fluorescence microscopy is performed using a Zeiss LSM 510 confocal microscope and Anti alphal Na/K ATPase mAb (Upstate/ Millipore, Billerica, Mass.) to further confirm the internalization of surface Na/K ATPase.

APN: APN abundance is measured both by Western analysis and QIHC using a polyclonal rabbit anti APN Ab (Santa Cruz Biotechnology, Santa Cruz, Calif.). APN activity is measured in cell membranes using fluorogenic substrate Arg-Phe-AFC (Enzyme System Products) by virtue of the fluorescence of its hydrolysis product.

Inhibition of APN and AT4 receptor: Complementary studies are conducted with highly specific siRNA to APN and to AT4 receptors. siRNA to APN (5′-UCGGGCACAGAUCAUUAUUd(TT)-3′; SEQ ID NO: 3) and to the amino terminal of the AT4 receptor (5′-AACCTGAGTCAGGACGTAAT-3′; SEQ ID NO: 4) have been designed for 95% efficacy (Qiagen Primer Design) is transfected into cell lines using the Qiagen Transmessenger kit with OPTIMEM media (Life technologies, Gaithersburg Md.). Reduction of APN and AT4 receptor abundance is confirmed by immunoblotting. As a control for APN siRNA, reversal of effects with siRNA is sought with co-transfection with the APN transgene. A second control is to measure AT4 receptor abundance in cells transfected with siRNA to APN. In complementary experiments, cells are treated with the APN inhibitor bestatin [(2S,3R)-3-amino-2-hydroxy-4-phenylbutanoyl-L-leucine] ((Ki) of 1.9×10⁻⁸). Since it is a relatively non-specific inhibitor and may affect distal Ang IV signaling independent of APN, control experiments are performed in which regulation of Na/K ATPase by Ang IV is tested.

Ang IV/divalinal-Ang IV treatment: Cells are incubated (4 hours, 37° C.) with Ang IV or divalinal Ang IV (1 nM-10 μM) (Kd=3.0+/−0.2 nmol/L; Bmax=508+/−30 fmol/mg protein for divalinal-Ang IV AT4 receptor binding).

Monoclonal and polyclonal Abs to Ang IV developed can be used as neutralizing Ang IV in cultured cells for complementrary studies. If so, complementary studies are conducted in which the effect of inhibiting Ang IV signaling using Ab is measured.

MAPK: Activities of P38 MAPK, Erk-1/2 are measured by quantification of phosphoprotein using specific primary monoclonal antibodies (New England Biolabs, Beverly, Mass.) and a commercially available kit (Cell Signaling Technology, Beverly, Mass.) in subconfluent cells either overexpressing APN or treated with siRNA to APN.

p38MAPK inhibitors SB20358 and MEK/Erk-1/2 inhibitor PD 98059 from Cell Signaling (Beverly, Mass.), are also used in recombinant cultured cells to implicate p38MAPK signaling in APN and Ang IV mediated reduction in basolateral Na/K ATPase expression. For overexpression of Erk and p38MAPK, full length cDNA clones for each (Open Biosystems), is cloned into pcDNA3.1 (Invitrogen). For AT1 receptor antagonist, candesartan is used.

Cultured Cells: siRNA to APN will increase and APN overexpression will reduce, respectively, the activity and abundance of plasmalemmal Na/K ATPase. Ang IV will reduce, respectively, activity and abundance of plasmalemmal Na/K ATPase. Exogenous and divalinal Ang IV will reduce regulation of Na/K ATPase by APN. siRNA to AT4 (IRAP) will block regulation of Na/K ATPase by APN and exogenous Ang IV. Over-expression of APN will lead to Ang IV signaling, i.e.: Activation of Erk-1 and P38 MAPK activation; and, SB20358 and PD 98059, inhibitors of p38MAPK and Erk -1/2 respectively, will inhibit the reduction in Na/K ATPase associated with APN over-expression.

Dahl rats: SR rats: Ang IV will reduce proximal tubule Na/KATPase activity to a greater extent in rats on 0.3% versus 8% NaCl.

SS rats: Ang IV will reduce proximal tubule Na/KATPase activity to the same extent,

and similar to that in SR rats on 0.3% NaCl, in rats on 0.3% versus 8% NaCl.

Data Analysis: Cultured Cells: The results of these experiments address the hypothesis that APN inhibits basolateral Na/K ATPase and that the mechanism is via Ang IV signaling.

APN: If siRNA to APN increases and over-expression of APN reduces the basolateral Na/K ATPase expression respectively, this supports our hypothesis that APN signaling regulates basolateral Na/K ATPase expression. If there are discordant results, e.g., siRNA does not increase or over-expression of APN decreases basolateral Na/K ATPase expression, this suggests that endogenous expression of APN in the cells is too low to regulate basolateral expression of Na/K ATPase or endogenous APN signaling is at a maximum, respectively. These possibilities will be resolved from results of experiments in which endogenous APN is inhibited with siRNA and recombinant APN is subsequently induced.

Ang IV: If siRNA to APN reduces and over-expression of APN increases Ang IV this suggests that APN regulates Ang IV and supports our hypothesis. If siRNA does not reduce Ang IV and/or APN over-expression does not increase Ang IV, then experiments are conducted to show the possibilities that Ang III substrate is rate limiting in the cells, and/or that 2) metabolism or efflux of Ang IV is regulated by APN. To address these possibilities, we will use exogenously administered Ang III and Ang IV.

Role of Ang IV signaling in APN-mediated reduction in basolateral Na/KATPase expression. If siRNA to the AT4 receptor (IRAP) attenuates the reduction in basolateral Na/K ATPase expression with APN over-expression, this suggests that signaling is through the AT4 receptor. If there is not a decrease, this suggests that signaling is not Ang IV/AT4 receptor dependent.

Role of conversion of Ang III to Ang IV by APN: If addition of Ang III reduces Na/K ATPase expression and siRNA to APN prevents this, this suggests that conversion of Ang III to Ang IV by APN is linked to regulation of Na/K ATPase. If the addition of Ang IV to these cells reduces Na/K ATPase expression, this provides further evidence. If Ang III does not reduce Na/K ATPase expression, this suggests that either Ang III conversion by APN is not playing a role or Ang III activation of AT1 receptors is counterbalancing effects of AT4 receptor activation. If cells treated with candasartan to block AT1 receptors show increased Na/K ATPase expression, it suggests that the latter is occurring.

Role of Erk-1 and p38 MAPK: If Erk-1 and/or p38 MAPK are activated during and inhibition of these kinases prevent APN mediated reduction in basolateral Na/K ATPase expression, this suggests that APN signaling is through these endocytotic pathways. If inhibition of these kinases does not prevent APN-mediated reduction of basolateral Na/K ATPase, expression, this suggests that the Erk-1 and p38 MAPK endocytotic pathway are not involved. Alternate signaling pathways that are implicated in regulation of Na/K ATPase would then be considered, e.g., enkaphalins, specifically dynorphin-A, since these are both metabolized by APN and have been linked to inhibition of cardiac Na/K ATPase.

Proximal tubule cells from Dahl rats: If Ang IV reduces Na/K ATPase activity in Dahl SS and SR rats on 0.3% NaCl, this suggests that signaling by the AT4 receptor is intact. If it does not decrease activity in either strain, this indicates that the AT4 receptor or signal distal to it is altered in the rat strain. If Ang IV does not reduce Na/K ATPase activity in Dahl SS rats on 8% NaCl, this indicates that reduced Ang IV does not play a role in greater Na/K ATPase activity in the Dahl SS rat.

What is claimed is:

1. A method of detecting, diagnosing and differentiating between salt-sensitive hypertension, salt-resistant hypertension, salt-independent hypertension and/or hypertensive disorders comprising:
detecting at least one or more biomarkers in a subject sample, and; correlating the detection of one or more biomarkers with a salt-sensitive hypertension, salt-resistant hypertension, salt-independent hypertension and/or hypertensive disorders wherein the correlation takes into account the detection of one or more biomarker in each diagnosis, as compared to normal subjects wherein the one or more markers are selected from:
APN, Ang II, Ang III, Ang IV, Ang 1-7, Anpep, aminopeptidase A (APA), AT4, Na/K ATPase, aldosterone, angiotensinogen, Mas and ACE2, fragments and variants thereof;
correlating the detection of one or more protein biomarkers with a diagnosis and differentiation between salt-sensitive hypertension, salt-resistant hypertension, salt-independent hypertension and/or hypertensive disorders, wherein the correlation takes into account the detection of one or more protein biomarkers in each diagnosis, as compared to normal subjects.

2. The method of claim 1, wherein the biomarkers further comprise metabolites of APN, Ang II, Ang III, Ang IV, Ang 1-7, Anpep, aminopeptidase A (APA), AT4, Na/K ATPase, angiotensinogen and ACE2.

3. The method of claim 1, wherein biomarkers distinguishing salt- resistant hypertension from salt- independent and salt- sensitive hypertension comprise: APN, Ang II, Ang IV, Anpep, AT4 and Ang 1-7.

4. The method of claim 3, wherein expression of biomarkers APN, Ang IV, Anpep, AT4 Ang 1-7 Ang II, Ang III, and APA are modulated as compared to biomarkers detected in salt-sensitive hypertension.

5. The method of claim 1, wherein a plurality of the biomarkers are detected.

6. The method of claim 1, wherein at least one of the biomarkers is detected.

7. The method of claim 1, wherein the sample is selected from the group consisting of saliva, sputum, breath condensate, blood, blood plasma, serum, urine, tissue, cells, kidney and liver.

8. The method of claim 1, wherein the sample is urine and/or plasma.

9. The method of claim 1, wherein biomarkers detected in a patient are elevated as compared to a normal healthy control.

10. The method of claim 1, wherein any combination of at least two biomarkers are used for detecting, diagnosing and differentiating between salt-sensitive hypertension, salt-resistant hypertension, salt-independent hypertension and/or hypertensive disorders.

11. The method of claim 1, wherein a plurality of the markers are used in combination for detecting, diagnosing and differentiating between salt-sensitive hypertension, salt-resistant hypertension, salt-independent hypertension and/or hypertensive disorders.

12. The method of claim 1, wherein one or more protein biomarkers are detected by comparing biomarker profiles from patients susceptible to, or suffering from salt-sensitive hypertension, salt-resistant hypertension, salt-independent hypertension and/or hypertensive disorders, with normal subjects.

13. The method of claim 1, wherein one or more protein biomarkers are detected using a fluorogenic assay immunoassay.

14. The method of claim 1, wherein one or more protein biomarkers are detected using a biochip array.

15. A method of diagnosing salt-sensitive hypertension in a subject, comprising measuring at least one of: APN, Ang III, Ang IV, Ang 1-7, Anpep, aminopeptidase A (APA), AT4, Na/K ATPase, aldosterone, angiotensinogen, Mas and ACE2, fragments, variants and metabolites thereof, in a biological sample obtained from said subject, and,
comparing levels of APN, Ang IV, Anpep, AT4 and Ang 1-7, fragments, variants and metabolites thereof, to normal, salt-resistant and salt independent hypertensive subjects; and,
correlating the detection of one or more protein biomarkers with a diagnosis and differentiation between salt-sensitive hypertension, salt-resistant hypertension, salt-independent hypertension and/or hypertensive disorders, wherein the correlation takes into account the detection of one or more protein biomarkers in each diagnosis, as compared to normal subjects.

16. A method of monitoring effectiveness of treatment of salt-sensitive hypertension comprising measuring at least one of: APN, Ang III, Ang IV, Ang 1-7, Anpep, aminopeptidase A (APA), AT4, Na/K ATPase, aldosterone, angiotensinogen, Mas and ACE2 in a biological sample obtained from said subject, wherein detected levels of APN, Ang III, Ang IV, Ang 1-7, Anpep, aminopeptidase A (APA), AT4, Na/K ATPase, aldosterone, angiotensinogen, Mas and ACE2 compared to normal subjects is indicative of the effectiveness of treatment of salt-sensitive hypertension.

17. A kit for diagnosing and differentiating between salt-sensitive hypertension, salt-resistant hypertension, salt-independent hypertension and/or hypertensive disorders in a subject, the kit comprising:
(a) a substrate for holding a biological sample isolated from a human subject suspected of having hypertension,
(b) a colorimetric agent that detects at least one or more biomarkers;
(c) a panel of biomarkers; and,
(d) printed instructions for reacting the agent with the biological sample or a portion of the biological sample to detect the presence or amount of at least one marker in the biological sample.

18. The kit of claim 17, wherein the panel of biomarkers comprise any one or more of: APN, Ang III, Ang IV, Ang 1-7, Anpep, aminopeptidase A (APA), AT4, Na/K ATPase, aldosterone, angiotensinogen, Mas and ACE2, metabolites and variants thereof.

19. The kit of claim 17, wherein the kit further comprises antibodies specific for any one or more biomarkers: APN, Ang III, Ang IV, Ang 1-7, Anpep, aminopeptidase A (APA), AT4, Na/K ATPase, aldosterone, angiotensinogen, Mas and ACE2, metabolites and variants thereof.

20. A method of identifying patients at risk of developing salt-sensitive hypertension comprising:
detecting at least one or more biomarkers in a subject sample, and; correlating the detection of one or more biomarkers with a diagnosis of salt-sensitive hypertension, wherein the correlation takes into account the detection of one or more biomarker in patients, as compared to normal subjects wherein the one or more markers are selected from:
APN, Ang III, Ang IV, Ang 1-7, Anpep, aminopeptidase A (APA), AT4, Na/K ATPase, aldosterone, angiotensinogen, Mas and ACE2, metabolites and variants thereof;
correlating the detection of one or more protein biomarkers with at risk patients for development of salt-sensitive hypertension, wherein the correlation takes into account the detection of one or more protein biomarkers in each at risk patient, as compared to normal subjects.

21. A panel of biomarkers comprising one or more biomarkers selected from:
APN, Ang III, Ang IV, Ang 1-7, Anpep, aminopeptidase A (APA), AT4, Na/K ATPase, aldosterone, angiotensinogen, Mas and ACE2, metabolites and variants thereof.

22. A method of identifying therapeutically effective doses of a pharmaceutical agent for treating hypertension in a subject, comprising measuring at least one of: APN, Ang III, Ang IV, Ang 1-7, Anpep, aminopeptidase A (APA), AT4, Na/K ATPase, aldosterone, angiotensinogen, Mas and ACE2, metabolites and variants thereof in a biological sample obtained from said subject, wherein an elevated or decreased level of APN, Ang III, Ang IV, Ang 1-7, Anpep, aminopeptidase A (APA), AT4, Na/K ATPase, aldosterone, angiotensinogen, Mas and ACE2, metabolites and variants thereof, compared to normal subjects is predictive of a decrease in hypertension and identifies the therapeutic effective dose.
